(19) United States
(12) Patent Application Publication
(10) Pub. No.: US 2024/0285625 A1
(43) Pub. Date: Aug. 29, 2024

(54) ERK1/2 AND KRAS G12C INHIBITORS COMBINATION THERAPY

(71) Applicant: Erasca, Inc., San Diego, CA (US)

(72) Inventors: Robert Field SHOEMAKER, San Diego, CA (US); Erin Denise LEW, San Diego, CA (US); Wei LIN, San Diego, CA (US); Jingchuan ZHANG, San Diego, CA (US); Joanne OH, San Diego, CA (US)

(21) Appl. No.: 18/572,399

(22) PCT Filed: Jun. 23, 2022
(86) PCT No.: PCT/US2022/034684

§ 371 Date: Dec. 20, 2023

Related U.S. Application Data
(60) Provisional application No. 63/321,609, filed on Mar. 18, 2022.
(60) Provisional application No. 63/283,034, filed on Nov. 24, 2021.
(60) Provisional application No. 63/277,548, filed on Nov. 9, 2021.
(60) Provisional application No. 63/214,767, filed on Jun. 24, 2021.

(51) Int. Cl. A61K 31/506; A61K 31/519; A61K 45/06; A61P 35/00

(52) U.S. Cl. CPC A61K 31/506; A61K 31/519; A61K 45/06; A61P 35/00

(57) ABSTRACT

The present disclosure relates generally to the use of an ERK 1/2 inhibitor in combination with a KRAS G12C inhibitor for treating cancer, specifically solid tumors.

### CROSS-REFERENCE

This application claims the benefit of U.S. Provisional Application Ser. No. 63/214,767 filed Jun. 24, 2021, U.S. Provisional Application Ser. No. 63/277,548 filed Nov. 9, 2021, U.S. Provisional Application Ser. No. 63/283,034 filed Nov. 24, 2021, and U.S. Provisional Application Ser. No. 63/321,609 filed Mar. 18, 2022, which are hereby incorporated by reference in their entirety.

### BACKGROUND

ERK1 and ERK2 (collectively “ERK1/2”) are related protein-serine/threonine kinases that participate in, amongst others, the Ras-Raf-MEK-ERK signal transduction pathway, which is sometimes denoted as the mitogen-activated protein kinase (MAPK) pathway. This pathway is thought to play a central role in regulating a number of fundamental cellular processes including one or more of cell proliferation, survival, adhesion, cycle progression, migration, differentiation, metabolism, and transcription. The activation of the MAPK pathway has been reported in numerous tumor types including lung, colon, pancreatic, renal, and ovarian cancers. Accordingly, substances that could reduce activation could be of interest for possible treatments.

### SUMMARY

ERK 1/2 appear to be activated by MEK through phosphorylation of both a threonine and a tyrosine residue, namely at Tyr204/187 and Thr202/185. Once activated, ERK1/2 catalyze the phosphorylation of serine/threonine residues of more than 100 substrates and activate both cytosolic and nuclear proteins that are linked to cell growth, proliferation, survival, angiogenesis and differentiation, all hallmarks of the cancer phenotype. Thus it may be beneficial to target ERK 1 and ERK 2 to develop and use ERK1/2 inhibitors as a way to inhibit tumor growth.

Furthermore, an ERK inhibitor may have utility in combination with other kinase, for example MAPK, inhibitors. Recently, researchers reported that dual inhibition of MEK and ERK by small molecule inhibitors was synergistic and acted to overcome acquired resistance to MEK inhibitors. See Hatzivassiliou et al., ERK Inhibition Overcomes Acquired Resistance to MEK Inhibition, Mol. Cancer Ther. 2012, 11, 1143-1154.

In addition to ERK 1/2, the RAS-MAPK signal transduction pathway includes the Ras family of proteins. The family includes three related GTPases (K-, N- and HRAS) that play a role in signal transduction pathways. KRAS, in particular, is known to have numerous mutations indicating an oncogenic state. KRAS mutants, such as mutations occurring at amino acid residue 12 (i.e., G12X), are commonly known to cause cancer. For example, the G12C mutation occurs in about 13% of NSCLC patients, and 1% to 3% of colorectal cancer and solid tumors.

The present embodiments disclosed herein generally relate to compositions and methods related to combination therapies to treat cancer utilizing an ERK1/2 inhibitor in conjunction with a KRAS-G12C inhibitor while providing an unexpected degree of synergy.

Disclosed herein is a method of treating cancer in a subject in need thereof, the method comprising: administering to the subject in need thereof a therapeutically effective amount of

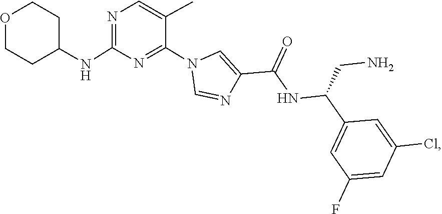 [SMILES: Cc1cnc(NC2CCOCC2)nc1-n1cnc(C(=O)N[C@H](CN)c2cc(F)cc(Cl)c2)c1]

(i) compound 1: or a pharmaceutically acceptable salt thereof; and
(ii) a KRAS G12C inhibitor.

In some embodiments, the KRAS G12C inhibitor is adagrasib, ARS-3248, BBP-454, BI 1701963, GDC-6036, sotorasib, or tipifarnib.

In some embodiments, the KRAS G12C inhibitor is sotorasib.

In some embodiments, sotorasib is administered in an amount that is about 960 mg/day.

In some embodiments, the KRAS G12C inhibitor is adagrasib.

In some embodiments, adagrasib is administered in an amount that is about 1200 mg/day.

Also disclosed herein is a method of treating cancer in a subject in need thereof, the method comprising: administering to the subject in need thereof a therapeutically effective amount of
(i) compound 1:

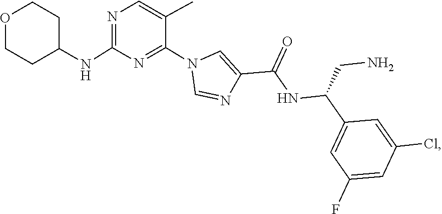 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) sotorasib.

Also disclosed herein is a method of treating cancer in a subject in need thereof, the method comprising: administering to the subject in need thereof a therapeutically effective amount of
(i) compound 1:

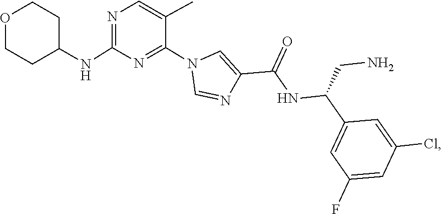 [SMILES: Cc1cnc(NC2CCOCC2)nc1-n1cnc(C(=O)N[C@H](CN)c2cc(F)cc(Cl)c2)c1]

acceptable salt thereof; and or a pharmaceutically
(ii) adagrasib.

In some embodiments, the pharmaceutically acceptable salt of compound 1 is the mandelic acid salt.

In some embodiments, the cancer is a mitogen-activated protein kinase (MAPK) pathway driven cancer.

In some embodiments, the cancer is a BRAF-driven cancer. HRAS-driven cancer, or a NRAS-driven cancer.

In some embodiments, the cancer comprises at least one cancer cell driven by deregulated ERK.

In some embodiments, the cancer has at least one mutation in RAS. In some embodiments, the cancer has at least one mutation in RAF. In some embodiments, the cancer has at least one mutation in MEK.

In some embodiments, the cancer has a G12C KRAS mutation. In some embodiments, the cancer has a G12D KRAS mutation. In some embodiments, the cancer has a G12S KRAS mutation. In some embodiments, the cancer has a G12V KRAS mutation. In some embodiments, the cancer has a G13D KRAS mutation. In some embodiments, the cancer has a Q16H KRAS mutation. In some embodiments, the cancer has a Q16K KRAS mutation. In some embodiments, the cancer has a Q6IR NRAS mutation.

In some embodiments, the cancer is a BRAF V600E or V600K mutant tumor.

In some embodiments, the cancer is a MAPKm/MAPKi-naïve pan cancer.

In some embodiments, the cancer comprises one or more EGFR mutation selected from the group consisting of EGFR gene copy gain. EGFR gene amplification, chromosome 7 polysomy. L858R, cxon 19 deletions/insertions. L86IQ. G719C. G719S. G719A. V765A. T783A, cxon 20 insertions. EGFR splice variants (Viii. Vvi, and Vii), A289D. A289T. A289V. G598A. G598V. T790M, and C797S.

In some embodiments, the cancer comprises one or more EGFR mutation selected from the group consisting of L858R, exon 19 deletion, and T790M.

In some embodiments, the cancer is a solid tumor.

In some embodiments, the cancer is non-small cell lung cancer (NSCLC), melanoma, pancreatic cancer, salivary gland tumor, thyroid cancer, colorectal cancer (CRC), or esophageal cancer.

In some embodiments, the cancer is non-small cell lung cancer (NSCLC). In some embodiments, the NSCLC is an EGFR mutant NSCLC. In some embodiments, the NSCLC is a KRAS G12C mutant NSCLC. In some embodiments, the NSCLC is a KRAS G12D mutant NSCLC. In some embodiments, the NSCLC is a KRAS G12S mutant NSCLC. In some embodiments, the NSCLC is a KRAS G12V mutant NSCLC. In some embodiments, the NSCLC is a KRAS G13D mutant NSCLC. In some embodiments, the NSCLC is a KRAS Q61H mutant NSCLC. In some embodiments, the NSCLC is a KRAS Q61K mutant NSCLC. In some embodiments, the NSCLC is a NRAS Q61R mutant NSCLC. In some embodiments, the cancer is a MAPKm/MAPKi-naïve NSCLC. In some embodiments, the cancer is a BRAFi-treated V600 NSCLC. In some embodiments, the cancer is a KRAS-treated G12C NSCLC. In some embodiments, the cancer is a KRAS-treated G12D NSCLC. In some embodiments, the cancer is a KRAS-treated G12S NSCLC. In some embodiments, the cancer is a KRAS-treated G12V NSCLC. In some embodiments, the cancer is a KRAS-treated G13D NSCLC. In some embodiments, the cancer is a KRAS-treated Q61H NSCLC. In some embodiments, the cancer is a KRAS-treated Q61K NSCLC. In some embodiments, the cancer is a NRAS-treated Q61R NSCLC.

In some embodiments, the cancer is pancreatic cancer.

In some embodiments, the cancer is a MAPKm/MAPKi-naïve pancreatic cancer.

In some embodiments, the cancer is melanoma.

In some embodiments, the melanoma is a BRAF V600E or V600K mutant tumor.

In some embodiments, the cancer is a BRAFi-treated V600 melanoma.

In some embodiments, the cancer is salivary gland tumor.

In some embodiments, the cancer is thyroid cancer.

In some embodiments, the cancer is colorectal cancer (CRC). In some embodiments, the CRC is a BRAF V600E CRC. In some embodiments, the CRC is a KRAS mutant CRC. In some embodiments. the CRC is a KRAS G12C mutant CRC. In some embodiments, the CRC is a KRAS G12D mutant CRC. In some embodiments, the CRC is a KRAS G12S mutant CRC. In some embodiments, the CRC is a KRAS G12V mutant CRC. In some embodiments, the CRC is a KRAS G13D mutant CRC. In some embodiments, the CRC is a KRAS Q61H mutant CRC. In some embodiments, the CRC is a KRAS Q61K mutant CRC. In some embodiments, the CRC is a NRAS mutant CRC. In some embodiments, the CRC is a NRAS Q61R mutant CRC.

In some embodiments, the cancer is esophageal cancer.

In some embodiments, compound 1, or a pharmaceutically acceptable salt thereof, is administered in an amount that is between about 25 mg/day and about 300 mg/day.

In some embodiments, compound 1, or a pharmaceutically acceptable salt thereof, is administered in an amount that is between 25 mg/day and 150 mg/day.

In some embodiments, compound 1, or a pharmaceutically acceptable salt thereof, is administered in an amount that is about 25 mg/day, about 50 mg/day, about 75 mg/day, about 100 mg/day, about 125 mg/day about 150 mg/day, about 175 mg/day, about 200 mg/day, about 225 mg/day, or about 250 mg/day.

In some embodiments, compound 1, or a pharmaceutically acceptable salt thereof, is administered in an amount that is about 25 mg/day, about 50 mg/day, about 100 mg/day, or about 150 mg/day.

In some embodiments, compound 1, or a pharmaceutically acceptable salt thereof, is administered in an amount that is about 250 mg/day.

In some embodiments, compound 1, or a pharmaceutically acceptable salt thereof, is administered once a day (QD). In some embodiments, compound 1, or a pharmaceutically acceptable salt thereof, is administered twice a day (BID). In some embodiments, compound 1, or a pharmaceutically acceptable salt thereof, is administered three times a day (TID).

In some embodiments, compound 1, or a pharmaceutically acceptable salt thereof, is administered once a week. In some embodiments, compound 1, or a pharmaceutically acceptable salt thereof, is administered twice a week.

In some embodiments, compound 1, or a pharmaceutically acceptable salt thereof, is administered in an amount that is between about 25 mg and about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is about 25 mg, 50 mg, about 75 mg, about 100 mg, about 125 mg, about 150 mg, about 175 mg, about 200 mg, about 225 mg, or about 250 mg twice a day, once a week (BID-QW).

In some embodiments, compound 1, or a pharmaceutically acceptable salt thereof, is administered in an amount that is about 25 mg. 50 mg, about 100 mg, about 125 mg, or about 150 mg twice a day, once a week (BID-QW).

In some embodiments, compound 1, or a pharmaceutically acceptable salt thereof, is administered in an amount that is about 125 mg twice a day, once a week (BID-QW).

In some embodiments, compound 1, or a pharmaceutically acceptable salt thereof, is administered for at least one 28-day cycle.

In some embodiments, compound 1, or a pharmaceutically acceptable salt thereof, is administered on day 1, day 8, day 15, and day 22 of a 28-day cycle.

In some embodiments, compound 1, or a pharmaceutically acceptable salt thereof, is administered on day 1, day 8, day 15 of a 28-day cycle.

In some embodiments, compound 1, or a pharmaceutically acceptable salt thereof, is administered orally.

In some embodiments, the method further comprises administering an additional MAPK pathway inhibitor.

In some embodiments, the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFRIR inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor.

In some embodiments, the additional MAPK pathway inhibitor is a KRAS inhibitor.

In some embodiments, the additional MAPK pathway inhibitor is a BRAF inhibitor.

In some embodiments, the additional MAPK pathway inhibitor is an EGFR inhibitor.

In some embodiments, the additional MAPK pathway inhibitor is a CDK4/6 inhibitor.

In some embodiments, the additional MAPK pathway inhibitor is a FLT3 inhibitor.

In some embodiments, the additional MAPK pathway inhibitor is adagrasib, afatinib, ASTX029, binimctinib, cetuximab, cobimetinib, dabrafenib, dacomitinib, encorafenib, erlotinib, gefitinib, gilteritinib, lapatinib, LTT462. LY3214996, necitumumab, neratinib, nimotuzumab, osimertinib, palbociclib, panitumumab, selumetinib, sotorasib, tramctinib, ulixertinib, and vandetanib.

In some embodiments, the additional MAPK pathway inhibitor is cctuximab.

In some embodiments, the additional MAPK pathway inhibitor is dabrafenib.

In some embodiments, the additional MAPK pathway inhibitor is encorafenib.

In some embodiments, the additional MAPK pathway inhibitor is gilteritinib.

In some embodiments, the additional MAPK pathway inhibitor is palbociclib.

In some embodiments, the additional MAPK pathway inhibitor is panitumumab.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1A shows cell viability assay data for Compound 1 and sotorasib in NCI-H2122-GFP cells.

FIG. 1B shows cell viability assay data for Compound 1 and sotorasib in HCC1171-GFP cells.

FIG. 1C shows cell viability assay data for Compound 1 and sotorasib in LU65 cells.

FIG. 1D shows cell viability assay data for Compound 1 and sotorasib in NCI-H23-GFP cells.

FIG. 1E shows cell viability assay data for Compound 1 and sotorasib in HCC44-GFP cells.

FIG. 1F shows cell viability assay data for Compound 1 and sotorasib in MIA PaCa-2-GFP cells.

FIG. 1G shows cell viability assay data for Compound 1 and sotorasib in NCI-H2030 cells.

FIG. 1H shows cell viability assay data for Compound 1 and sotorasib in LU99 cells.

FIG. 1I shows cell viability assay data for Compound 1 and sotorasib in LIM2099 cells.

FIG. 1J shows cell viability assay data for Compound 1 and sotorasib in SW837 cells.

FIG. 2A shows cell viability assay data for Compound 1 and adagrasib in NCI-H2122-GFP cells.

FIG. 2B shows cell viability assay data for Compound 1 and adagrasib in HCC1171-GFP cells.

FIG. 2C shows cell viability assay data for Compound 1 and adagrasib in LU65 cells.

FIG. 2D shows cell viability assay data for Compound 1 and adagrasib in NCI-H23-GFP cells.

FIG. 2E shows cell viability assay data for Compound 1 and adagrasib in HCC44-GFP cells.

FIG. 2F shows cell viability assay data for Compound 1 and adagrasib in MIA PaCa-2-GFP cells.

FIG. 2G shows cell viability assay data for Compound 1 and adagrasib in NCI-H2030 cells.

FIG. 2H shows cell viability assay data for Compound 1 and adagrasib in LU99 cells.

FIG. 2I shows cell viability assay data for Compound 1 and adagrasib in LIM2099 cells.

FIG. 2J shows cell viability assay data for Compound 1 and adagrasib in SW837 cells.

FIG. 3 shows that Compound 1 and sotorasib demonstrate combination benefit in vivo in KRASG12C mutant CRC PDX model CO-04-0307 based on exemplary tumor growth curves (PDX=Patient-derived xenograft).

FIG. 4 shows that Compound 1 and sotorasib demonstrate combination benefit in vivo in KRASG12C mutant CRC PDX model CO-04-0310 based on exemplary tumor growth curves.

FIG. 5 shows that Compound 1 and sotorasib demonstrate combination benefit in vivo in KRAS G12C mutant NSCLC PDX model LU-01-0046 based on exemplary tumor growth curves.

### DETAILED DESCRIPTION

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein. “consist of” or “consist essentially of” the described features.

As used in the specification and appended claims, unless specified to the contrary, the following terms have the meaning indicated below.

As used herein, the term “therapeutic” means an agent utilized to treat, combat, ameliorate, prevent or improve an unwanted condition or disease of a patient. In some embodiments, a therapeutic agent such as a compound 1 is directed to the treatment and/or the amelioration of cancers.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering”, when used in conjunction with a composition described herein, can include, but is not limited to, providing a composition into or onto the target tissue: providing a composition systemically to a patient by, e.g., oral administration whereby the therapeutic reaches the target tissue or cells. “Administering” a composition may be accomplished by injection, topical administration, and oral administration or by other methods alone or in combination with other known techniques.

The term “animal” as used herein includes, but is not limited to, humans and non-human vertebrates such as wild, domestic and farm animals. As used herein, the terms “patient.” “subject” and “individual” are intended to include living organisms in which certain conditions as described herein can occur. Examples include humans, monkeys, cows, sheep, goats, dogs, cats, mice, rats, and transgenic species thereof. In a preferred embodiment, the patient is a primate. In certain embodiments, the primate or subject is a human. In certain instances, the human is an adult. In certain instances, the human is child. In further instances, the human is under the age of 12 years. In certain instances, the human is elderly. In other instances, the human is 60 years of age or older. Other examples of subjects include experimental animals such as mice, rats, dogs, cats, goats, sheep, pigs, and cows. The experimental animal can be an animal model for a disorder, e.g., a transgenic mouse with hypertensive pathology.

By “pharmaceutically acceptable”, it is meant the carrier, diluent or excipient must be compatible with the other ingredients of the formulation and not deleterious to the recipient thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease: for example, preventing a disease, condition or disorder in an individual that may be predisposed to the disease, condition or disorder but does not yet experience or display the pathology or symptomatology of the disease. (2) inhibiting the disease: for example, inhibiting a disease, condition or disorder in an individual that is experiencing or displaying the pathology or symptomatology of the disease, condition or disorder (i.e., arresting further development of the pathology and/or symptomatology), and (3) ameliorating the disease: for example, ameliorating a disease, condition or disorder in an individual that is experiencing or displaying the pathology or symptomatology of the disease, condition or disorder (i.e., reversing the pathology and/or symptomatology).

The terms “treat.” “treated.” “treatment.” or “treating” as used herein refers to both therapeutic treatment in some embodiments and prophylactic or preventative measures in other embodiments, wherein the object is to prevent or slow (lessen) an undesired physiological condition, disorder or disease, or to obtain beneficial or desired clinical results. For the purposes described herein, beneficial or desired clinical results include, but are not limited to, alleviation of symptoms: diminishment of the extent of the condition, disorder or disease: stabilization (i.e., not worsening) of the state of the condition, disorder or disease: delay in onset or slowing of the progression of the condition, disorder or disease: amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

The term “substantially the same as” as used herein, refers to a powder x-ray diffraction pattern or differential scanning calorimetry pattern that is non-identical to those depicted herein, but that falls within the limits of experimental error, when considered by one of ordinary skill in the art.

### Compound 1

Disclosed herein is (S)—N-(2-amino-1-(3-chloro-5-fluorophenyl)ethyl)-1-(5-methyl-2-((tetrahydro-2H-pyran-4-yl)amino)pyrimidin-4-yl)-1H-imidazole-4-carboxamide:

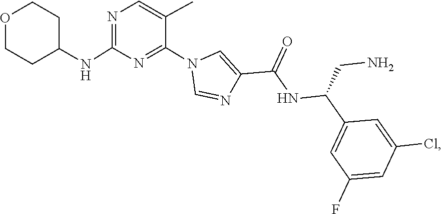 [SMILES: Cc1cnc(NC2CCOCC2)nc1-n1cnc(C(=O)N[C@H](CN)c2cc(F)cc(Cl)c2)c1]

or a pharmaceutically acceptable salt thereof.

In some embodiments, the salt of compound 1 is the mandelic acid salt. In some embodiments, the salt of compound 1 is the benzenesulfonic acid salt. In some embodiments, the salt of compound 1 is the hydrochloride salt. In some embodiments, the salt of compound 1 is the p-toluenesulfonic acid salt. In some embodiments, the salt of compound 1 is the benzenesulfonic acid salt.

### KRAS G12C Inhibitors

KRAS is a key regulator of signaling pathway responsible for cell proliferation, differentiation, and survival. KRAS is the most frequently mutated oncogene in human cancer and mutations in KRAS can results in continuous cellular proliferation and cancer development. The G12C mutation is a single point mutation with a glycine-to-cysteine substitution at codon 12. This substitution favors the activated state of KRAS, amplifying signaling pathways that lead to oncogenesis.

In some embodiments, the KRAS G12C inhibitor is adagrasib, ARS-3248, BBP-454, BI 1701963, GDC-6036, sotorasib, or tipifarnib.

In some embodiments, the KRAS G12C inhibitor is adagrasib. In some embodiments, the KRAS G12C inhibitor is sotorasib.

### Sotorasib

Sotorasib,

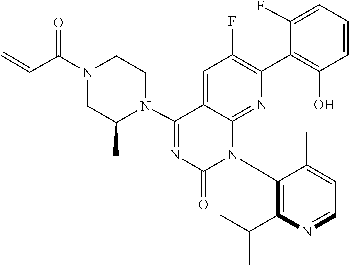

sold under the brand names Lumakras® and Lumykras®, is sold by Amgen and is an anti-cancer medication used to treat non-small-cell lung cancer (NSCLC). It targets a specific mutation, G12C, in the protein K-Ras encoded by gene KRAS which is responsible for various forms of cancer. Sotorasib is an inhibitor of the RAS GTPase family.

Sotorasib is the first approved targeted therapy for tumors with any KRAS mutation, which accounts for approximately 25% of mutations in non-small cell lung cancers. KRAS G12C mutations occur in about 13% of patients with non-small cell lung cancers.

In May 2021, sotorasib was approved by the FDA for the treatment of KRAS G12C mutated NSCLC.

### Adagrasib

Adagrasib (MRTX-849)

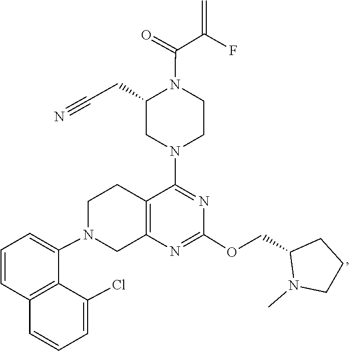 [SMILES: C=C(F)C(=O)N1CCN(c2nc(OC[C@@H]3CCCN3C)nc3c2CCN(c2cccc4cccc(Cl)c24)C3)C[C@@H]1CC#N]

is an experimental cancer drug being developed by Mirati Therapeutics. It acts as a covalently binding inhibitor for a mutant form of the protein KRAS called G12C, which is commonly present in various forms of cancer and acts as a growth factor. It has shown promising results in pre-clinical testing and is currently in clinical trials.

### Combinations

Disclosed herein is a method of treating cancer in a subject in need thereof, the method comprising administering to the subject in need thereof a therapeutically effective amount of
(i) compound 1:

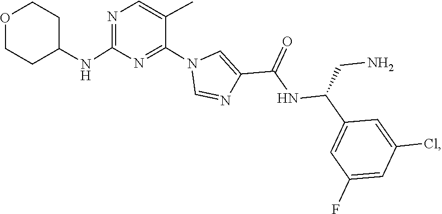 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) a KRAS G12C inhibitor.

Disclosed herein is a method of treating cancer in a subject in need thereof, the method comprising administering to the subject in need thereof a therapeutically effective amount of
(i) compound 1:

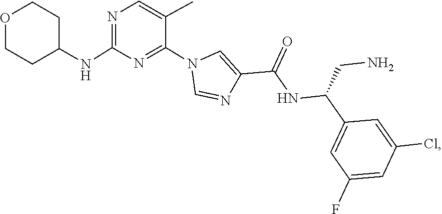 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) sotorasib.

Disclosed herein is a method of treating cancer in a subject in need thereof, the method comprising administering to the subject in need thereof a therapeutically effective amount of
(i) compound 1:

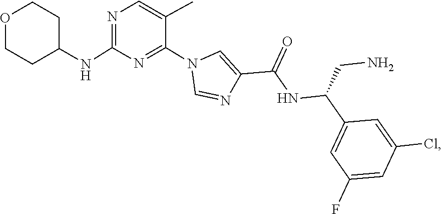 [SMILES: Cc1cnc(NC2CCOCC2)nc1-n1cnc(C(=O)N[C@H](CN)c2cc(F)cc(Cl)c2)c1]

acceptable salt thereof; and or a pharmaceutically
(ii) adagrasib.

### Further Combination

In some embodiments, the method comprises administering an additional MAPK pathway inhibitor. Without being bound by theory, suppression of MAPK signaling in cancer cells can result in downregulation of PD-LI expression and increase the likelihood that the cancer cells are detected by the immune system. Such third MAPK pathway inhibitors may be based on other mutations of proteins in the MAPK pathway. In some embodiments, the additional MAPK pathway inhibitor inhibits a protein in the MAPK pathway. In some embodiments, the additional MAPK pathway inhibitor inhibits a protein outside the MAPK pathway. In some embodiments, the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFRIR inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor. Exemplary MAPK pathway inhibitors include, without limitation, adagrasib, afatinib, ASTX029, binimetinib, cetuximab, cobimetinib, dabrafenib, dacomitinib, encorafenib, erlotinib, gefitinib, gilteritinib, lapatinib, LTT462, LY3214996, necitumumab, neratinib, nimotuzumab, osimertinib, palbociclib, panitumumab, selumetinib, sotorasib, trametinib, ulixertinib, and vandetanib.

In some embodiment the additional MAPK pathway inhibitor is adagrasib. In some embodiment the additional MAPK pathway inhibitor is afatinib. In some embodiment the additional MAPK pathway inhibitors is binimctinib. In some embodiment the additional MAPK pathway inhibitor is cetuximab. In some embodiment the additional MAPK pathway inhibitor is cobimetinib. In some embodiment the additional MAPK pathway inhibitor is dabrafenib. In some embodiment the additional MAPK pathway inhibitor is dacomitinib. In some embodiment the additional MAPK pathway inhibitor is encorafenib. In some embodiment the additional MAPK pathway inhibitor is erlotinib. In some embodiment the additional MAPK pathway inhibitor is gefitinib. In some embodiment the additional MAPK pathway inhibitor is gilteritinib. In some embodiment the additional MAPK pathway inhibitor is lapatinib. In some embodiment the additional MAPK pathway inhibitor is LTT462. In some embodiment the additional MAPK pathway inhibitor is LY3214996. In some embodiment the additional MAPK pathway inhibitor is necitumumab. In some embodiment the additional MAPK pathway inhibitor is neratinib. In some embodiment the additional MAPK pathway inhibitor is nimotuzumab. In some embodiment the additional MAPK pathway inhibitor is osimertinib. In some embodiment the additional MAPK pathway inhibitor is palbociclib. In some embodiment the additional MAPK pathway inhibitor is panitumumab. In some embodiment the additional MAPK pathway inhibitor is selumctinib. In some embodiment the additional MAPK pathway inhibitor is sotorasib. In some embodiment the additional MAPK pathway inhibitor is trametinib. In some embodiment the additional MAPK pathway inhibitor is ulixertinib. In some embodiment the additional MAPK pathway inhibitor is vandetanib.

### Cancers

Disclosed herein are methods of treating cancer using a combination disclosed herein. “Cancer” refers to all types of cancer, neoplasm or malignant tumors found in mammals (e.g. humans), including, without limitation, leukemias, lymphomas, myelomas, carcinomas, and sarcomas. Exemplary cancers that may be treated with a compound or method provided herein include brain cancer, glioma, glioblastoma, neuroblastoma, prostate cancer, colorectal cancer, pancreatic cancer (such as pancreatic adenocarcinoma, PDAC), medulloblastoma, melanoma, cervical cancer, gastric cancer, ovarian cancer, lung cancer, cancer of the head. Hodgkin's Disease, and Non-Hodgkin's Lymphomas. Exemplary cancers that may be treated with a compound or method provided herein include cancer of the blood, thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

In some embodiments, the cancer has a class 1 B-Raf mutation.

In some embodiments, the cancer harbors at least one of a EGFR, KRAS, BRAF (e.g., BRAF class III) and/or NF1 (e.g., loss of function) mutations.

In some embodiments, the mutant B-Raf comprises a V600 mutation. In some embodiments, the mutant of B-Raf comprises the mutation V600E. In some embodiments, the mutation is V600K. In some embodiments, the mutation is V600D. In some embodiments, the mutation is V600L. In some embodiments, the mutation is V600R. In some embodiments, the cancer is a BRAF V600E or V600K mutant tumor.

In some embodiments, the cancer is a mitogen-activated protein kinase (MAPK) pathway driven cancer.

In some embodiments, the cancer is a BRAF-driven cancer, HRAS-driven cancer, or a NRAS-driven cancer.

In some embodiments, the cancer comprises at least one cancer cell driven by deregulated ERK.

In some embodiments, the cancer has at least one mutation in RAS. In some embodiments, the cancer has at least one mutation in RAF. In some embodiments, the cancer has at least one mutation in MEK.

In some embodiments, the cancer has a G12C KRAS mutation. In some embodiments, the cancer has a G12D KRAS mutation. In some embodiments, the cancer has a G12R KRAS mutation. In some embodiments, the cancer has a G12S KRAS mutation. In some embodiments, the cancer has a G12V KRAS mutation. In some embodiments, the cancer has a G12W KRAS mutation. In some embodiments, the cancer has a G13D KRAS mutation. In some embodiments, the cancer has a H95D KRAS mutation. In some embodiments, the cancer has a H95Q KRAS mutation. In some embodiments, the cancer has a H95R KRAS mutation. In some embodiments, the cancer has a Q61H KRAS mutation. In some embodiments, the cancer has a G12D KRAS mutation. In some embodiments, the cancer has a Q61K KRAS mutation. In some embodiments, the cancer has a Q61R NRAS mutation. In some embodiments, the cancer has a R68S KRAS mutation.

In some embodiments, the cancer is a MAPKm/MAPKi-naïve pan cancer.

In some embodiments, the cancer comprises one or more EGFR mutation selected from the group consisting of EGFR gene copy gain. EGFR gene amplification, chromosome 7 polysomy, L858R. exon 19 deletions/insertions, L861Q, G719C, G719S, G719A, V765A, T783A, exon 20 insertions, EGFR splice variants (Viii, Vvi, and Vii), A289D, A289T, A289V. G598A, G598V, T790M, and C797S. In some embodiments, the cancer comprises one or more EGFR mutation selected from the group consisting of L858R, exon 19 deletion, and T790M.

In some embodiments, the cancer is a solid tumor. In some embodiments, the solid tumor is an advanced or a metastatic solid tumor.

In some embodiments, the cancer is non-small cell lung cancer (NSCLC), melanoma, pancreatic cancer, salivary gland tumor, thyroid cancer, colorectal cancer (CRC), or esophageal cancer.

In some embodiments, the cancer is colorectal cancer (CRC), pancreatic ductal adenocarcinoma (PDAC), cholangiocarcinoma cancer, appendiceal cancer, gastric cancer, esophageal cancer, non-small cell lung cancer (NSCLC), head and neck cancer, ovarian cancer, uterine cancer, acute myeloid leukemia (AML), or melanoma.

In some embodiments, the cancer is a gastrointestinal cancer. In some embodiments, the gastrointestinal is anal cancer, bile duct cancer, colon cancer, rectal cancer, esophageal cancer, gallbladder cancer, liver cancer, pancreatic cancer, small intestine cancer, or stomach cancer (gastric cancer).

In some embodiments, the cancer is non-small cell lung cancer (NSCLC). In some embodiments, the NSCLC is an EGFR mutant NSCLC. In some embodiments, the NSCLC is a KRAS G12C mutant NSCLC. In some embodiments, the NSCLC is a KRAS G12D mutant NSCLC. In some embodiments, the NSCLC is a KRAS G12S mutant NSCLC. In some embodiments, the NSCLC is a KRAS G12V mutant NSCLC. In some embodiments, the NSCLC is a KRAS G13D mutant NSCLC. In some embodiments, the NSCLC is a KRAS Q61H mutant NSCLC. In some embodiments, the NSCLC is a KRAS Q61K mutant NSCLC. In some embodiments, the NSCLC is a KRAS G12R mutant NSCLC. In some embodiments, the NSCLC is a KRAS G12W mutant NSCLC. In some embodiments, the NSCLC is a KRAS H95D mutant NSCLC. In some embodiments, the NSCLC is a KRAS H95Q mutant NSCLC. In some embodiments, the NSCLC is a KRAS H95R mutant NSCLC. In some embodiments, the NSCLC is a KRAS G12D mutant NSCLC. In some embodiments, the NSCLC is a KRAS R68S mutant NSCLC.

In some embodiments, the NSCLC is a NRAS Q61R mutant NSCLC. In some embodiments, the cancer is a MAPKm/MAPKi-naïve NSCLC. In some embodiments, the cancer is a BRAFi-treated V600 NSCLC. In some embodiments, the cancer is a KRAS-treated G12C NSCLC. In some embodiments, the cancer is a KRAS-treated G12D NSCLC. In some embodiments, the cancer is a KRAS-treated G12S NSCLC. In some embodiments, the cancer is a KRAS-treated G12V NSCLC. In some embodiments, the cancer is a KRAS-treated G13D NSCLC. In some embodiments, the cancer is a KRAS-treated Q61H NSCLC. In some embodiments, the cancer is a KRAS-treated Q61K NSCLC. In some embodiments, the cancer is a NRAS-treated Q61R NSCLC. In some embodiments, the cancer is a KRAS-treated G12R NSCLC. In some embodiments, the cancer is a KRAS-treated G12W NSCLC. In some embodiments, the cancer is a KRAS-treated H95D NSCLC. In some embodiments, the cancer is a KRAS-treated H95Q NSCLC. In some embodiments, the cancer is a KRAS-treated H95R NSCLC. In some embodiments, the cancer is a KRAS-treated G12D NSCLC. In some embodiments, the cancer is a KRAS-treated R68S NSCLC.

In some embodiments, the cancer is pancreatic cancer. In some embodiments, the cancer is a MAPKm/MAPKi-naïve pancreatic cancer. In some embodiments, the cancer is pancreatic ductal adenocarcinoma (PDAC). In some embodiments, the PDAC cancer has a G12V mutation.

In some embodiments, the cancer is melanoma. In some embodiments, the melanoma is a BRAF V600E or V600K mutant tumor. In some embodiments, the cancer is a BRAFi-treated V600 melanoma.

In some embodiments, the cancer is salivary gland tumor.

In some embodiments, the cancer is thyroid cancer.

In some embodiments, the cancer is colorectal cancer (CRC). In some embodiments, the CRC is a BRAF V600E CRC. In some embodiments, the CRC is a KRAS mutant CRC.

In some embodiments, the CRC is a KRAS G12C mutant CRC. In some embodiments, the CRC is a KRAS G12D mutant CRC. In some embodiments, the CRC is a KRAS G12S mutant CRC. In some embodiments, the CRC is a KRAS G12V mutant CRC. In some embodiments, the CRC is a KRAS G13D mutant CRC. In some embodiments, the CRC is a KRAS Q61H mutant CRC. In some embodiments, the CRC is a KRAS Q61K mutant CRC. In some embodiments, the CRC is a NRAS mutant CRC. In some embodiments, the CRC is a NRAS Q61R mutant CRC.

In some embodiments, the cancer is esophageal cancer.

In some embodiments, the cancer has one or more acquired mutations. In some embodiments, the acquired mutation results from a first-line treatment. In some embodiments, the first-line treatment is a KRAS inhibitor. In some embodiments, the KRAS inhibitor is a KRAS G12C inhibitor. In some embodiments, the KRAS G12C inhibitor is adagrasib. In some embodiments, the KRAS G12C inhibitor is sotorasib. In some embodiments, the cancer is a solid tumor cancer. In some embodiments, the cancer is NSCLC.

In some embodiments, the acquired mutation is an acquired KRAS mutation. In some embodiments, the acquired mutation is KRAS G12C. In some embodiments, the acquired mutation is KRAS G12D. In some embodiments, the acquired mutation is KRAS G12R. In some embodiments, the acquired mutation is KRAS G12V. In some embodiments, the acquired mutation is KRAS G12W. In some embodiments, the acquired mutation is KRAS G13D. In some embodiments, the acquired mutation is KRAS H95D. In some embodiments, the acquired mutation is KRAS H95D. In some embodiments. the acquired mutation is KRAS H95Q. In some embodiments, the acquired mutation is KRAS H95R. In some embodiments, the acquired mutation is KRAS Q61H. In some embodiments, the acquired mutation is KRAS R68S.

In some embodiments, the acquired mutation is an acquired MAPK pathway mutation. In some embodiments, the acquired MAPK pathway mutation is MAP2K1 K57N. In some embodiments, the acquired MAPK pathway mutation is MAP2K1 K57T. In some embodiments, the acquired MAPK pathway mutation is CCDC6-RET. In some embodiments, the acquired MAPK pathway mutation is RITI P128L. In some embodiments, the acquired MAPK pathway mutation is PTEN G209V. In some embodiments, the acquired MAPK pathway mutation is BRAF V600E. In some embodiments, the acquired MAPK pathway mutation is MAP2K1 199_K104del. In some embodiments, the acquired MAPK pathway mutation is MAP2K1 K57N. In some embodiments, the acquired MAPK pathway mutation is EML4-ALK. In some embodiments, the acquired MAPK pathway mutation is EGFR A289A. In some embodiments, the acquired MAPK pathway mutation is FGFR3-TACC3. In some embodiments. the acquired MAPK pathway mutation is AKAP9-BRAF. In some embodiments, the acquired MAPK pathway mutation is RAF1-CCDC176. In some embodiments, the acquired MAPK pathway mutation is RAF1-TRAKI. In some embodiments, the acquired MAPK pathway mutation is NRAS Q61K. In some embodiments, the acquired MAPK pathway mutation is MAP2K1 E102_1103 DEL. In some embodiments, the acquired MAPK pathway mutation is NRF1-BRAF.

In some embodiments, the acquired mutation is a KRAS G12C reactivation mutation. In some embodiments, the KRAS G12C reactivation mutation is a RKRAS G12C gene amplification. In some embodiments, the KRAS G12C reactivation mutation is a NF1 R22637 (LoF).

In some embodiments, the acquired mutation is a non-G12C activation KRAS mutation. In some embodiments, the non-G12C activation KRAS mutation is KRAS G12D. In some embodiments. the non-G12C activation KRAS mutation is KRAS G12R. In some embodiments, the non-G12C activation KRAS mutation is KRAS G12V. In some embodiments, the non-G12C activation KRAS mutation is KRAS G12W. In some embodiments, the non-G12C activation KRAS mutation is KRAS G13D. In some embodiments, the non-G12C activation KRAS mutation is KRAS Q61H. In some embodiments, the non-G12C activation KRAS mutation is KRAS Q61K.

In some embodiments, the acquired mutation is a sterically hindering KRAS G12C mutation. In some embodiments, the sterically hindering KRAS G12C mutation is KRAS R68S. In some embodiments, the sterically hindering KRAS G12C mutation is KRAS H95D. In some embodiments, the sterically hindering KRAS G12C mutation is KRAS H95Q. In some embodiments, the sterically hindering KRAS G12C mutation is KRAS H95R. In some embodiments, the sterically hindering KRAS G12C mutation is KRAS Y96C.

In some embodiments, the acquired mutation is an RTK activation mutation. In some embodiments, the RTK activation mutation is EGFR A289V. In some embodiments, the RTK activation mutation is RET M918T. In some embodiments, the RTK activation mutation is MET genc amplification. In some embodiments, the RTK activation mutation is EML-ALK. In some embodiments, the RTK activation mutation is CCDC6-RET. In some embodiments, the RTK activation mutation is FGFR3-TACC3.

In some embodiments, the acquired mutation is a downstream RAS/MAPK activation mutation. In some embodiments, the downstream RAS/MAPK activation mutation is BRAF V600E. In some embodiments, the downstream RAS/MAPK activation mutation is MAP2K 199_K104del. In some embodiments, the downstream RAS/MAPK activation mutation is MAP2K1 199_K104del. In some embodiments, the downstream RAS/MAPK activation mutation is MAP2K1 E102_1103 del. In some embodiments, the downstream RAS/MAPK activation mutation is RAF fusion.

In some embodiments, the acquired mutation is a parallel pathway activation mutation. In some embodiments, the parallel pathway activation mutation is PIK3CA H1047R. In some embodiments, the parallel pathway activation mutation is PIK3R1 S361fs. In some embodiments, the parallel pathway activation mutation is PTEN N48K. In some embodiments, the parallel pathway activation mutation is PTEN G209V. In some embodiments, the parallel pathway activation mutation is RITI P128L.

### Dosing

In one aspect, the compositions described herein are used for the treatment of diseases and conditions described herein. In addition, a method for treating any of the diseases or conditions described herein in a subject in need of such treatment, involves administration of compositions in therapeutically effective amounts to said subject.

Dosages of compositions described herein can be determined by any suitable method. Maximum tolerated doses (MTD) and maximum response doses (MRD) for compound 1, or a pharmaceutically acceptable salt thereof can be determined via established animal and human experimental protocols as well as in the examples described herein. For example, toxicity and therapeutic efficacy of compound 1, or a pharmaceutically acceptable salt thereof, can be determined by standard pharmaceutical procedures in cell cultures or experimental animals, including, but not limited to, for determining the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. The data obtained from cell culture assays and animal studies can be used in formulating a range of dosage for use in human. The dosage of such compounds lies preferably within a range of circulating concentrations that include the EDso with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of a given formulation comprising compound 1, or a pharmaceutically acceptable salt thereof that corresponds to such an amount varies depending upon factors such as the molecular weight of a particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

In some embodiments, sotorasib is administered in an amount that is about 960 mg/day.

In some embodiments, adagrasib is administered in an amount that is about 1200 mg/day.

In some embodiments, the amount of compound 1, or a pharmaceutically acceptable salt thereof, as described herein is relative to the free-base equivalent of compound 1.

In some embodiments, compound 1, or a pharmaceutically acceptable salt thereof, is administered orally.

In some embodiments, compound 1, or a pharmaceutically acceptable salt thereof, is administered in an amount that is between about 25 mg/day and about 300 mg/day.

In some embodiments, compound 1, or a pharmaceutically acceptable salt thereof, is administered in an amount that is between 25 mg/day and 150 mg/day.

In some embodiments, compound 1, or a pharmaceutically acceptable salt thereof, is administered in an amount that is about 25 mg/day, about 50 mg/day, about 75 mg/day, about 100 mg/day, about 125 mg/day, about 150 mg/day, about 175 mg/day, about 200 mg/day, about 225 mg/day, or about 250 mg/day.

In some embodiments, compound 1, or a pharmaceutically acceptable salt thereof, is administered in an amount that is about 25 mg/day, about 50 mg/day, about 100 mg/day, or about 150 mg/day.

In some embodiments, compound 1, or a pharmaceutically acceptable salt thereof, is administered in an amount between about 25 mg to about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 200 mg twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is between about 25 mg and about 100 mg twice a day, once a week (BID-QW).

In some embodiments, compound 1, or a pharmaceutically acceptable salt thereof, is administered in an amount that is between about 25 mg and about 50 mg twice a day, once a week (BID-QW).

In some embodiments, compound 1, or a pharmaceutically acceptable salt thereof, is administered in an amount that is between about 50 mg to about 300 mg twice a day, once a week (BID-QW).

In some embodiments, compound 1, or a pharmaceutically acceptable salt thereof, is administered in an amount that is between about 50 mg and about 250 mg twice a day, once a week (BID-QW).

In some embodiments, compound 1, or a pharmaceutically acceptable salt thereof, is administered in an amount that is between about 50 mg and about 200 mg twice a day, once a week (BID-QW).

In some embodiments, compound 1, or a pharmaceutically acceptable salt thereof, is administered in an amount that is between about 50 mg and about 150 mg twice a day, once a week (BID-QW).

In some embodiments, compound 1, or a pharmaceutically acceptable salt thereof, is administered in an amount that is between about 50 mg and about 100 mg twice a day, once a week (BID-QW).

In some embodiments, compound 1, or a pharmaceutically acceptable salt thereof, is administered in an amount that is between about 100 mg and about 300 mg twice a day, once a week (BID-QW).

In some embodiments, compound 1, or a pharmaceutically acceptable salt thereof, is administered in an amount that is between about 100 mg and about 250 mg twice a day, once a week (BID-QW).

In some embodiments, compound 1, or a pharmaceutically acceptable salt thereof, is administered in an amount that is between about 100 mg and about 200 mg twice a day, once a week (BID-QW).

In some embodiments, compound 1, or a pharmaceutically acceptable salt thereof, is administered in an amount that is between about 100 mg and about 150 mg twice a day, once a week (BID-QW).

In some embodiments, compound 1, or a pharmaceutically acceptable salt thereof, is administered in an amount that is between about 150 mg and about 300 mg twice a day, once a week (BID-QW).

In some embodiments, compound 1, or a pharmaceutically acceptable salt thereof, is administered in an amount that is between about 150 mg and about 250 mg twice a day, once a week (BID-QW).

In some embodiments, compound 1, or a pharmaceutically acceptable salt thereof, is administered in an amount that is between about 150 mg and about 200 mg twice a day, once a week (BID-QW).

In some embodiments, compound 1, or a pharmaceutically acceptable salt thereof, is administered in an amount that is between about 175 mg and about 300 mg twice a day, once a week (BID-QW).

In some embodiments, compound 1, or a pharmaceutically acceptable salt thereof, is administered in an amount that is between about 175 mg and about 250 mg twice a day, once a week (BID-QW).

In some embodiments, compound 1, or a pharmaceutically acceptable salt thereof, is administered in an amount that is between about 175 mg and about 200 mg twice a day, once a week (BID-QW).

In some embodiments, compound 1, or a pharmaceutically acceptable salt thereof, is administered in an amount that is between about 200 mg and about 300 mg twice a day, once a week (BID-QW).

In some embodiments, compound 1, or a pharmaceutically acceptable salt thereof, is administered in an amount that is between about 200 mg and about 250 mg twice a day, once a week (BID-QW).

In some embodiments, compound 1, or a pharmaceutically acceptable salt thereof, is administered in an amount that is between about 225 mg and about 300 mg twice a day, once a week (BID-QW).

In some embodiments, compound 1, or a pharmaceutically acceptable salt thereof, is administered in an amount that is between about 225 mg and about 250 mg twice a day, once a week (BID-QW).

In some embodiments, compound 1, or a pharmaceutically acceptable salt thereof, is administered in an amount that is between about 25 mg and about 300 mg once a week (QW).

In some embodiments, compound 1, or a pharmaceutically acceptable salt thereof, is administered in an amount that is between about 50 mg and about 250 mg once a week (QW).

In some embodiments, compound 1, or a pharmaceutically acceptable salt thereof, is administered in an amount that is between about 100 mg and about 300 mg once a week (QW).

In some embodiments, compound 1, or a pharmaceutically acceptable salt thereof, is administered in an amount that is between about 100 mg and about 250 mg once a week (QW).

In some embodiments, compound 1, or a pharmaceutically acceptable salt thereof, is administered in an amount that is between about 150 mg and about 300 mg once a week (QW).

In some embodiments, compound 1, or a pharmaceutically acceptable salt thereof, is administered in an amount that is between about 150 mg and about 250 mg once a week (QW).

In some embodiments, compound 1, or a pharmaceutically acceptable salt thereof, is administered in an amount that is about 100 mg once a week (QW). In some embodiments, compound 1, or a pharmaceutically acceptable salt thereof, is administered in an amount that is about 150 mg once a week (QW). In some embodiments, compound 1, or a pharmaceutically acceptable salt thereof, is administered in an amount that is about 200 mg once a week (QW). In some embodiments, compound 1, or a pharmaceutically acceptable salt thereof, is administered in an amount that is about 250 mg once a week (QW).

In some embodiments, compound 1, or a pharmaceutically acceptable salt thereof, is administered in an amount that is between about 25 mg and about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is about 25 mg. 50 mg, about 75 mg, about 100 mg, about 125 mg, about 150 mg, about 175 mg, about 200 mg, about 225 mg, or about 250 mg twice a day, once a week (BID-QW).

In some embodiments, compound 1, or a pharmaceutically acceptable salt thereof, is administered in an amount that is about 25 mg. 50 mg, about 100 mg, about 125 mg, or about 150 mg twice a day, once a week (BID-QW).

In some embodiments, compound 1, or a pharmaceutically acceptable salt thereof, is administered in an amount that is about 125 mg twice a day, once a week (BID-QW).

In some embodiments, compound 1, or a pharmaceutically acceptable salt thereof, is administered in an amount that is about 250 mg once a day, once a week.

In some embodiments, compound 1, or a pharmaceutically acceptable salt thereof, is administered in an amount that is about 25 mg. 30 mg. 40 mg. 50 mg, about 60 mg, about 70 mg, about 75 mg, about 80 mg, about 90 mg, about 95 mg, about 100 mg, about 105 mg, about 110 mg, about 115 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 280 mg, about 290 mg, or about 300 mg.

In some embodiments, each of the above-recited amounts may be administered QD, QW, BID, BID-QD, or BID-QW.

### Administration

Administration of compound 1, or a pharmaceutically acceptable salt thereof, and combination partners described herein are at a dosage described herein or at other dose levels and compositions determined and contemplated by a medical practitioner. In certain embodiments, compound 1, or a pharmaceutically acceptable salt thereof, is administered for prophylactic and/or therapeutic treatments. In certain therapeutic applications, compound 1, or a pharmaceutically acceptable salt thereof, and combination partners described herein, are administered to a patient already suffering from a disease in an amount sufficient to cure the disease or at least partially arrest or ameliorate the symptoms. Amounts effective for this use depend on the age of the patient, severity of the disease, previous therapy, the patient's health status, weight, and response to the compositions, and the judgment of the treating physician. Therapeutically effective amounts are optionally determined by methods including, but not limited to, a dose escalation clinical trial.

In prophylactic applications, the compositions described herein are administered to a patient susceptible to or otherwise at risk of a particular disease, e.g., cancer. Such an amount is defined to be a “prophylactically effective amount or dose.” In this use, the precise amounts also depend on the patient's age, state of health, weight, and the like. When used in a patient, effective amounts for this use will depend on the risk or susceptibility of developing the particular disease, previous therapy, the patient's health status and response to the compositions, and the judgment of the treating physician.

In certain embodiments wherein the patient's condition does not improve, upon the doctor's discretion the administration of a composition described herein are administered chronically, that is, for an extended period of time, including throughout the duration of the patient's life in order to ameliorate or otherwise control or limit the symptoms of the patient's disease. In other embodiments, administration of a composition continues until complete or partial response of a disease.

In some embodiments, compound 1, or a pharmaceutically acceptable salt thereof, and

combination partners described herein, are administered once a day. In some embodiments, compound 1, or a pharmaceutically acceptable salt thereof, and combination partners described herein are administered twice a day. In some embodiments, compound 1, or a pharmaceutically acceptable salt thereof, and combination partners described herein are administered three times a day.

In some embodiments, sotorasib is administered once a day. In some embodiments, sotorasib is administered twice a day. In some embodiments, sotorasib is administered three times a day.

In some embodiments, adagrasib is administered once a day. In some embodiments, adagrasib is administered twice a day. In some embodiments, adagrasib is administered three times a day.

In some embodiments, compound 1, or a pharmaceutically acceptable salt thereof, and combination partners described herein are administered to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, compound 1, or a pharmaceutically acceptable salt thereof, is administered to a subject who is in a fasted state for at least 8 hours. In other embodiments, compound 1, or a pharmaceutically acceptable salt thereof, and combination partners described herein, are administered to a subject who is in a fasted state for at least 10 hours. In yet other embodiments, compound 1, or a pharmaceutically acceptable salt thereof, and combination partners described herein, are administered to a subject who is in a fasted state for at least 12 hours. In other embodiments, compound 1, or a pharmaceutically acceptable salt thereof, and combination partners described herein, are administered to a subject who has fasted overnight.

In other embodiments, compound 1, or a pharmaceutically acceptable salt thereof, and combination partners described herein, are administered to a subject who is in a fed state. A fed state refers to a subject who has taken food or has had a meal. In certain embodiments, a composition is administered to a subject in a fed state 5 minutes post-meal. 10 minutes post-meal. 15 minutes post-mcal. 20 minutes post-meal. 30 minutes post-meal. 40 minutes post-meal. 50 minutes post-meal. 1 hour post-meal, or 2 hours post-meal. In certain instances, compound 1, or a pharmaceutically acceptable salt thereof, is administered to a subject in a fed state 30 minutes post-meal. In other instances, compound 1, or a pharmaceutically acceptable salt thereof, and combination partners described herein, are administered to a subject in a fed state 1 hour post-meal. In yet further embodiments, compound 1, or a pharmaceutically acceptable salt thereof, is administered to a subject with food.

The length of a treatment cycle depends on the treatment being given. In some embodiments, the length of a treatment cycle ranges from two to six weeks. In some embodiments, the length of a treatment cycle ranges from three to six weeks. In some embodiments, the length of a treatment cycle ranges from three to four weeks. In some embodiments, the length of a treatment cycle is three weeks (or 21 days). In some embodiments, the length of a treatment cycle is four weeks (28 days). In some embodiments, the length of a treatment cycle is five weeks (35 days). In some embodiments, the length of a treatment cycle is 56 days. In some embodiments, a treatment cycle lasts one, two, three, four, or five weeks. In some embodiments, a treatment cycle lasts three weeks. In some embodiments, a treatment cycle lasts four weeks. In some embodiments, a treatment cycle lasts five weeks. The number of treatment doses scheduled within each cycle also varies depending on the drugs being given.

In some embodiments of a method of treating cancer, compound 1, or a pharmaceutically acceptable salt thereof, and combination partners described herein are administered in 28-day cycles. In some embodiments of a method of treating cancer, compound 1, or a pharmaceutically acceptable salt thereof, and combination partners described herein, are administered for multiple 28-day cycles. In some embodiments of a method of treating cancer, compound 1, or a pharmaceutically acceptable salt thereof, and combination partners described herein, are administered for at least one 28-day cycle. In some embodiments of a method of treating cancer, compound 1, or a pharmaceutically acceptable salt thereof, and combination partners described herein, are administered for at least two 28-day cycles. In some embodiments of a method of treating cancer, compound 1, or a pharmaceutically acceptable salt thereof, and combination partners described herein, are administered for at least three 28-day cycles. In some embodiments of a method of treating cancer, compound 1, or a pharmaceutically acceptable salt thereof, and combination partners described herein, are administered for at least four 28-day cycles. In some embodiments of a method of treating cancer, compound 1, or a pharmaceutically acceptable salt thereof, and combination partners described herein, are administered for at least five 28-day cycles. In some embodiments of a method of treating cancer, compound 1, or a pharmaceutically acceptable salt thereof, and combination partners described herein, are administered for at least six 28-day cycles.

In some embodiments of a method of treating cancer, compound 1, or a pharmaceutically acceptable salt thereof, is administered on days 1-7 of each 28-day cycle. In some embodiments of a method of treating cancer, compound 1, or a pharmaceutically acceptable salt thereof, is administered on days 1-14 of each 28-day cycle. In some embodiments of a method of treating cancer, compound 1, or a pharmaceutically acceptable salt thereof, is administered on days 1-21 of each 28-day cycle. In some embodiments of a method of treating cancer, compound 1, or a pharmaceutically acceptable salt thereof. is administered on days 1-28 of each 28-day cycle.

In some embodiments of a method of treating cancer, compound 1, or a pharmaceutically acceptable salt thereof, is administered twice a day on day 1 of a 28-day cycle. In some embodiments of a method of treating cancer, compound 1, or a pharmaceutically acceptable salt thereof, is administered twice a day on day 8 of a 28-day cycle. In some embodiments of a method of treating cancer, compound 1, or a pharmaceutically acceptable salt thereof, is administered twice a day on day 15 of a 28-day cycle. In some embodiments of a method of treating cancer, compound 1, or a pharmaceutically acceptable salt thereof, is administered twice a day on day 22 of a 28-day cycle. In some embodiments of a method of treating cancer, compound 1, or a pharmaceutically acceptable salt thereof, is not administered twice a day on day 22 of a 28-day cycle.

In some embodiments of a method of treating cancer, compound 1, or a pharmaceutically acceptable salt thereof, is administered twice a day on day 1, day 8, and day 15 of a 28-day cycle.

In some embodiments of a method of treating cancer, compound 1, or a pharmaceutically acceptable salt thereof, is not administered on days 2-7, days 9-14, days 16-21, days 23-28 of a 28-day cycle.

In some embodiments of a method of treating cancer, compound 1, or a pharmaceutically acceptable salt thereof, and combination partners described herein are administered in 35-day cycles. In some embodiments of a method of treating cancer, compound 1, or a pharmaceutically acceptable salt thereof, and combination partners described herein are administered for multiple 35-day cycles. In some embodiments of a method of treating cancer, compound 1, or a pharmaceutically acceptable salt thereof, and combination partners described herein are administered for at least one 35-day cycle. In some embodiments of a method of treating cancer, compound 1, or a pharmaceutically acceptable salt thereof, and combination partners described herein are administered for at least two 35-day cycle. In some embodiments of a method of treating cancer, compound 1, or a pharmaceutically acceptable salt thereof, and combination partners described herein are administered for at least three 35-day cycle. In some embodiments of a method of treating cancer, compound 1, or a pharmaceutically acceptable salt thereof, and combination partners described herein are administered for at least four 35-day cycle. In some embodiments of a method of treating cancer, compound 1, or a pharmaceutically acceptable salt thereof, and combination partners described herein are administered for at least five 35-day cycle. In some embodiments of a method of treating cancer, compound 1, or a pharmaceutically acceptable salt thereof, and combination partners described herein are administered for at least six 35-day cycle.

In some embodiments of a method of treating cancer, compound 1, or a pharmaceutically acceptable salt thereof, is administered on days 1-7 of each 35-day cycle. In some embodiments of a method of treating cancer, compound 1, or a pharmaceutically acceptable salt thereof, is administered on days 1-14 of each 35-day cycle. In some embodiments of a method of treating cancer, compound 1, or a pharmaceutically acceptable salt thereof, is administered on days 1-21 of each 35-day cycle. In some embodiments of a method of treating cancer, compound 1, or a pharmaceutically acceptable salt thereof. is administered on days 1-28 of each 35-day cycle. In some embodiments of a method of treating cancer, compound 1, or a pharmaceutically acceptable salt thereof, is administered on days 1-35 of each 35-day cycle.

In some embodiments of a method of treating cancer, compound 1, or a pharmaceutically acceptable salt thereof, is administered twice a day on day 1 of a 35-day cycle. In some embodiments of a method of treating cancer, compound 1, or a pharmaceutically acceptable salt thereof, is administered twice a day on day 8 of a 35-day cycle. In some embodiments of a method of treating cancer, compound 1, or a pharmaceutically acceptable salt thereof, is administered twice a day on day 15 of a 35-day cycle. In some embodiments of a method of treating cancer, compound 1, or a pharmaceutically acceptable salt thereof, is administered twice a day on day 22 of a 35-day cycle. In some embodiments of a method of treating cancer, compound 1, or a pharmaceutically acceptable salt thereof, is administered twice a day on day 29 of a 35-day cycle. In some embodiments of a method of treating cancer, compound 1, or a pharmaceutically acceptable salt thereof, is not administered twice a day on day 29 of a 35-day cycle.

In some embodiments of a method of treating a cancer, compound 1, or a pharmaceutically acceptable salt thereof, is administered twice a day on day 1, day 8, day 15, and day 22 of a 35-day cycle.

In some embodiments of a method of treating cancer, compound 1, or a pharmaceutically acceptable salt thereof, is not administered on days 2-7, days 9-14, days 16-21, days 23-28, and days 30-35 of a 28-day cycle.

### EXAMPLES

### Example 1: In-Vitro Viability Assay (Compound 1+Sotorasib)

Cells were plated at a density of 1.000 (NCI-H2122-GFP. LU65, NCI-H23-GFP. MIA PaCa-2-GFP, LU99, LIM2099) or 5.000 (HCC1171-GFP, HCC44, NCI-H2030. SW837) cells per well in a 96-well plate (Corning #3903). Cells were allowed to adhere overnight, and compound was added in a matrix format using a HP Tecan D300e digital dispenser (Switzerland). Compound 1 was added in a 1:3 dilution series (8-point dose response) from bottom to top of plate (rows B-H) and sotorasib was added in a 1:2 dilution series (11-point dose response) from right to left (columns 2-11). Final DMSO concentration was normalized across the plate. Cell viability was assessed 5-days post-treatment using

Promega CellTiter-Glo 3D Cell Viability Assay reagent (#G9683) according to manufacturer's protocol. Luminescence was assessed using a SpectraMax M3e (Molecular Devices, San Jose, CA) and combination benefit was assessed using the BLISS model in the Combenefit software (Cancer Research UK Cambridge Institute).

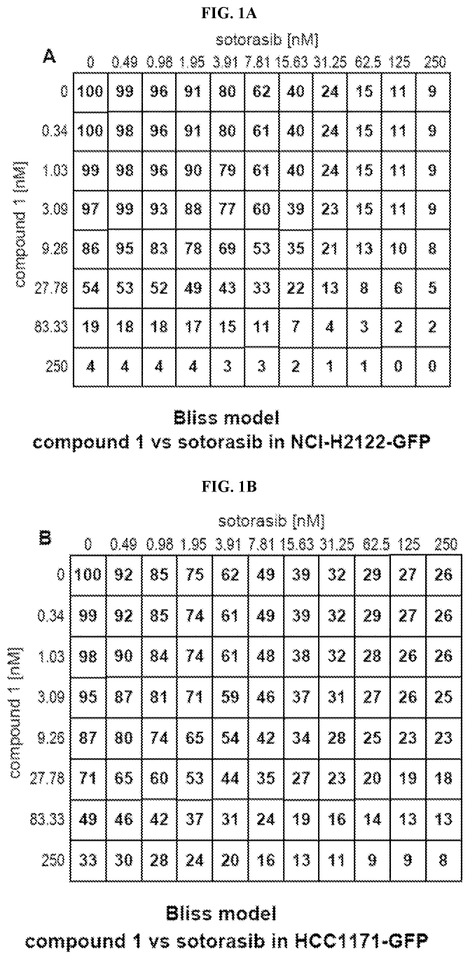

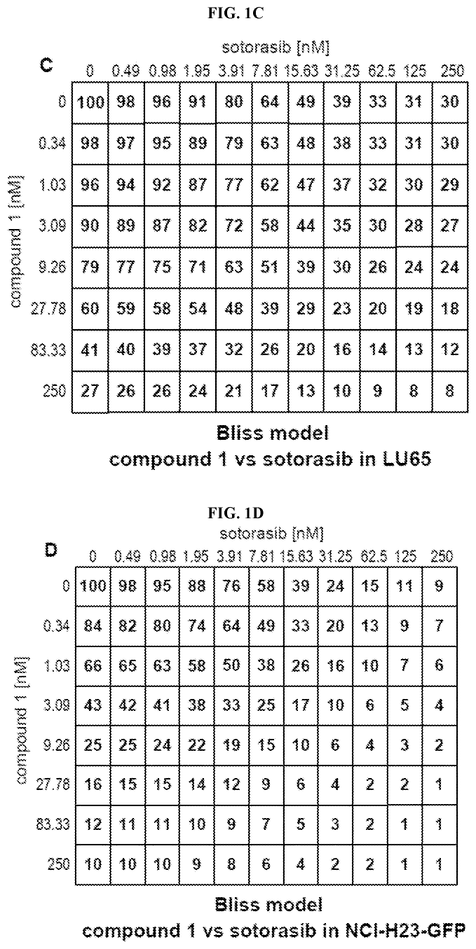

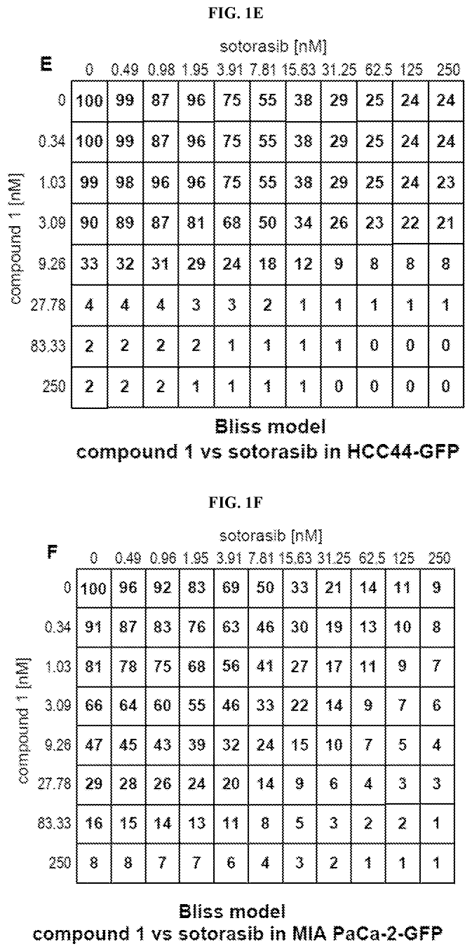

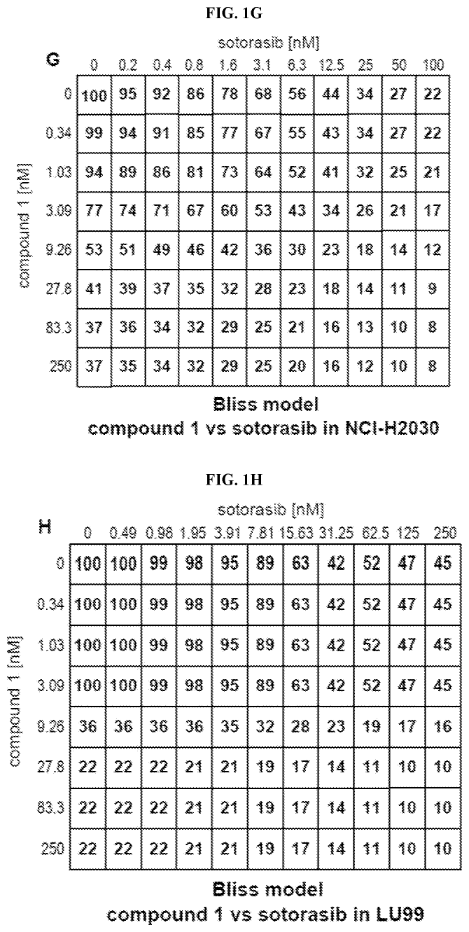

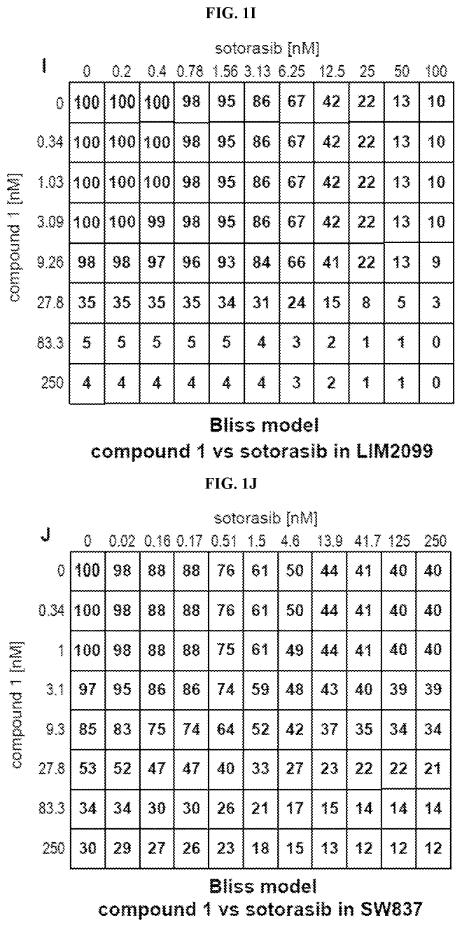

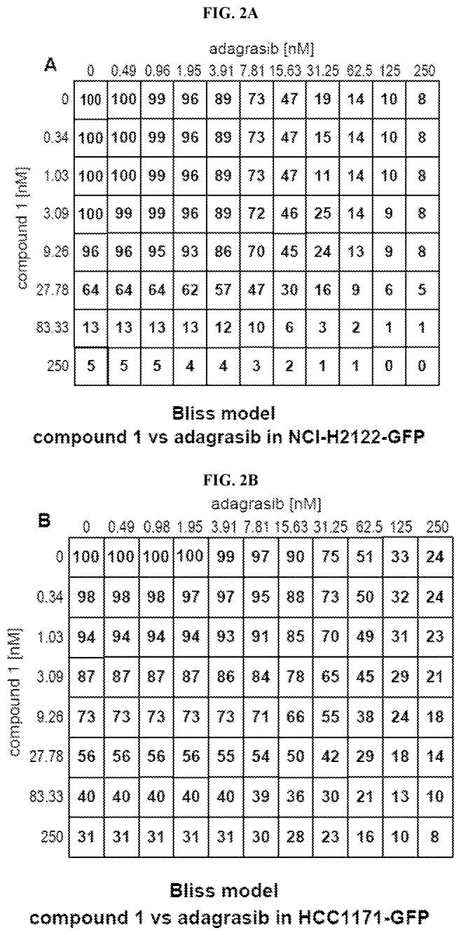

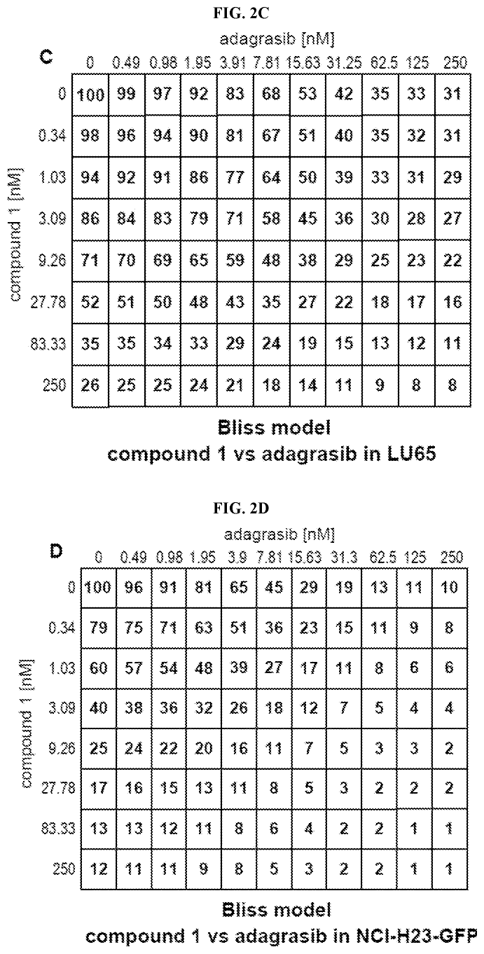

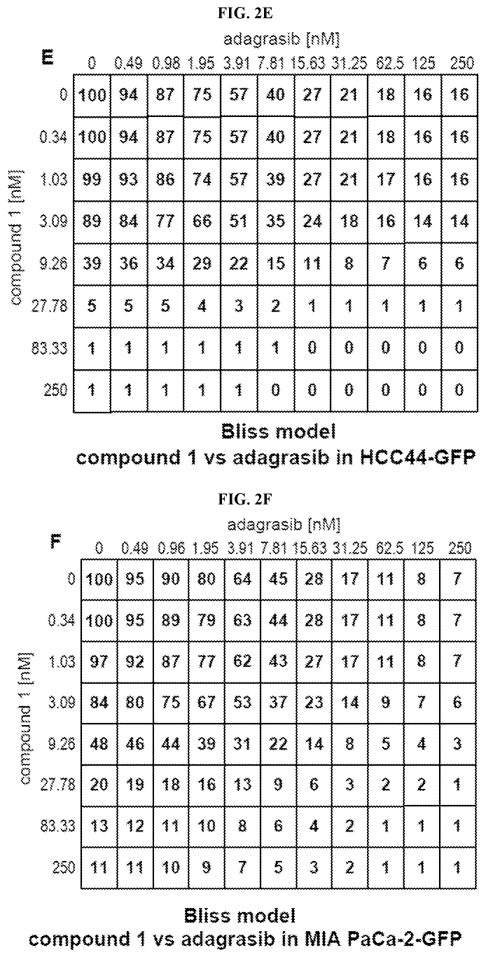

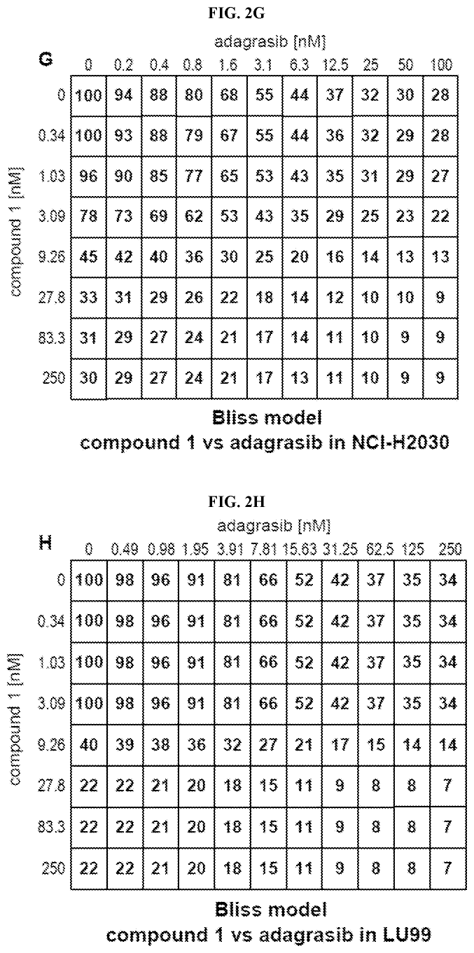

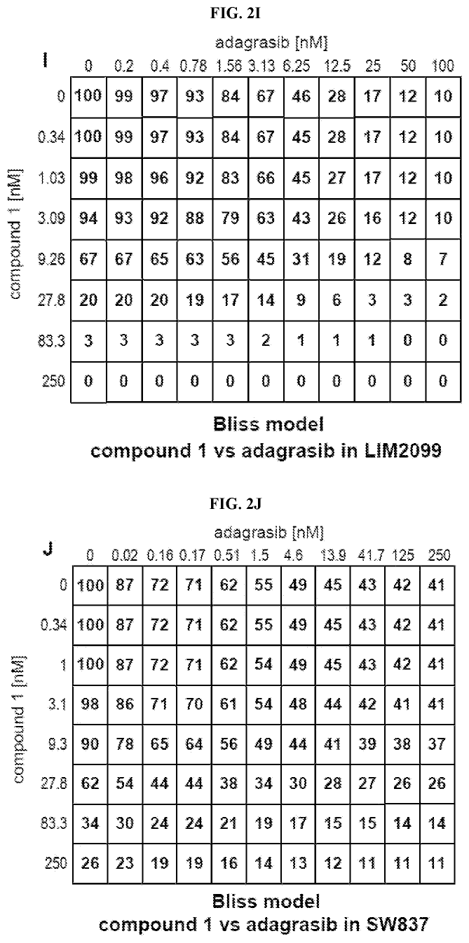

NCI-H2122-GFP cells (FIG. 1A), HCC1171-GFP cells (FIG. 1B). LU65 cells (FIG. 1C). NCI-H23-GFP cells (FIG. 1D), HCC44-GFP cells (FIG. 1E), MIA PaCa-2-GFP cells (FIG. 1F), NCI-H2030 cells (FIG. 1G). LU99 cells (FIG. 1H). LIM2099 cells (FIG. 1I), and SW837 cells (FIG. 1J) were treated with a dilution matrix of compound 1 vs sotorasib in 3D cell viability assays. Cell viability, as expressed as a percentage of viable cells relative to vehicle treated control, is shown in the matrix.

Compound 1 and sotorasib demonstrate combination activity in KRAS G12C cellular models.

### Example 2: In-Vitro Viability Assay (Compound 1+adagrasib)

Cells were plated at a density of 1.000 (NCI-H2122-GFP. LU65. NCI-H23-GFP. MIA PaCa-2-GFP, LU99, LIM2099) or 5.000 (HCC1171-GFP, HCC44, NCI-H2030, SW837) cells per well in a 96-well plate (Corning #3903). Cells were allowed to adhere overnight, and compound was added in a matrix format using a HP Tecan D300e digital dispenser (Switzerland). Compound 1 was added in a 1:3 dilution series (8-point dose response) from bottom to top of plate (rows B-H) and adagrasib was added in a 1:2 dilution series (11-point dose response) from right to left (columns 2-11). Final DMSO concentration was normalized across the plate. Cell viability was assessed 5-days post-treatment using Promega CellTiter-Glo 3D Cell Viability Assay reagent (#G9683) according to manufacturer's protocol. Luminescence was assessed using a SpectraMax M3c (Molecular Devices, San Jose, CA) and combination benefit was assessed using the BLISS model in the Combenefit software (Cancer Research UK Cambridge Institute).

NCI-H2122-GFP cells (FIG. 1A), HCC1171-GFP cells (FIG. 1B). LU65 cells (FIG. 1C), NCI-H23-GFP cells (FIG. 1D). HCC44-GFP cells (FIG. 1E), MIA PaCa-2-GFP cells (FIG. 1F), NCI-H2030 cells (FIG. 1G). LU99 cells (FIG. 1H). LIM2099 cells (FIG. 1I), and SW837 cells (FIG. 1J) were treated with a dilution matrix of compound 1 vs adagrasib in 3D cell viability assays. Cell viability, as expressed as a percentage of viable cells relative to vehicle treated control, is shown in the matrix.

Compound 1 and adagrasib demonstrate combination activity in KRAS G12C cellular models.

### Example 3: A Phase 1b/2 Study of Agents Targeting the Mitogen-Activated Protein Kinase Pathway in Patients with Advanced Non-Small-Cell Lung Cancer

This study will include: 1) the evaluation of the safety and tolerability of escalating doses of Compound 1 in combination with other cancer therapies in study participants with advanced non-small cell lung cancer (NSCLC): 2) the determination of the Maximum Tolerated Dose (MTD) and/or

Recommended Dosc (RD) of Compound 1 administered in combination with other cancer therapies: 3) the evaluation of the antitumor activity of Compound 1 in combination with other cancer therapies; and 4) the evaluation of the pharmacokinetic (PK) profiles of Compound 1 and other cancer therapies when administered in combination.

The Phase 1b/2 study will include evaluating safety, tolerability, and antitumor activity of Compound 1 in combination with other cancer therapies in study participants with advanced NSCLC. The study will include dose escalation cohorts in which Compound 1 plus sotorasib is administered to study participants with advanced NSCLC harboring Kirsten rat sarcoma G12C mutation (KRAS G12Cm). Compound 1 will be orally administered at multiple QW dose levels between 150 mg and 250 mg (inclusive) or BID-QW dose levels between 75 mg and 125 mg (inclusive) in combination with sotorasib to study participants with KRAS G12Cm NSCLC in sequential ascending doses until unacceptable toxicity, disease progression, or withdrawal of consent. Dose expansion will follow and will evaluate Compound 1 orally administered at the RD identified from the respective dose escalation cohort in study participants with advanced EGFRm or KRAS G12Cm NSCLC.

### Dosing Schedule

Compound 1 + Sotorasib in KRAS G12C NSCLC
Dose Escalation

| | |
|---|---|
| Compound 1 150 mg QW + Sotorasib | Compound 1 75 mg BID-QW + |
| | Sotorasib |
| Compound 1 200 mg QW + Sotorasib | Compound 1 100 mg BID-QW + |
| | Sotorasib |
| Compound 1 250 mg QW + Sotorasib | Compound 1 125 mg BID-QW + |
| | Sotorasib |

Dose Expansion

| | |
|---|---|
| Compound 1 QW (RD) + Sotorasib | Compound 1 BID-QW (RD) + |
| | Sotorasib |

The following inclusion and exclusion criteria will apply to the study:

### Inclusion Criteria

Age ≥18 years.

Willing and able to give written informed consent.

Have histologically or cytologically confirmed NSCLC, with presence of EGFR mutation(s) sensitive to EGFR inhibitors, or KRAS G12C mutation.

Measurable disease per Response Evaluation Criteria in Solid Tumors (RECIST) v1.1.

Adequate bone marrow and organ function.

Have Eastern Cooperative Oncology Group (ECOG) performance status of 0 or 1:
grade 0: fully active, able to carry on all pre-disease performance without restriction;
grade 1: restricted in physically strenuous activity but ambulatory and able to carry out work of a light or sedentary nature, e.g. light housework, office work.

Willing to comply with all protocol-required visits, assessments, and procedures.

Able to swallow oral medication.

### Exclusion Criteria

Concurrent treatment with any systemic anticancer therapy for NSCLC, including any approved or investigational agent.

For participants with KRAS G12Cm NSCLC: prior therapy with a SHP2, ERK, or KRAS G12C inhibitor (depending on which cohort is being considered for enrollment).

Palliative radiotherapy within 7 days of enrollment.

History of unacceptable toxicity to treatment with sotorasib.

Major surgery within the 28 days of enrollment.

Unresolved toxicities from prior systemic therapy greater than NCI Common Terminology Criteria for Adverse Events (CTCAE) grade 1 at time of enrollment, except for toxicities not considered a safety risk (e.g., alopecia, vitiligo, and grade 2 neuropathy due to prior chemotherapy).

History of another malignancy≤5 years prior to first dose, except for patients who are disease-free for >2 years after treatment with curative intent or who have carcinoma in situ.

Symptomatic and unstable brain metastases, or spinal cord compression, except for patients who have completed definitive therapy (surgery or radiotherapy), are not on steroids, and have a stable neurologic status for a least 2 weeks after completion of the definitive therapy and steroids.

History of or clinically active Interstitial Lung Disease (ILD), drug induced ILD, or radiation pneumonitis that required steroid treatment.

Impaired cardiovascular function or clinically significant cardiovascular disease.

History or current evidence of retinal pigment epithelial detachment (RPED), central serous retinopathy, retinal vein occlusion (RVO), or predisposing factors to RPED or RVO.

Any evidence of severe or uncontrolled systemic disease or evidence of any other significant clinical disorder or laboratory finding that renders the patient inappropriate to participate in the study.

Pregnant or breastfeeding women.

Contraindication to sotorasib use as per local label.

### Example 4: In Vivo KRASG12C Mutant CRC PDX model CO-04-0307 Assay

The vehicle/control article Propylene Glycol (PG) in deionized water, was prepared and stored under ambient conditions throughout the 28-day administration in mice.

Compound 1 was prepared in vehicle of 0.5% Methyl Cellulose (MC) and 0.1% Tween 80 solution weekly and stored under ambient conditions. The combination agent sotorasib was prepared weekly in vehicle of 50% Polyethylene Glycol 400+50% Propylene Glycol w/w, and stored at 2-8° C.

Female Balb/c nude mice were between 6-8 weeks of age at the time of implantation. Mice were hosted at a special pathogen-frec (SPF) environment of the vivarium facility and acclimated to their new environment for at least 3 days prior to initiation of any experiments according to IACUC protocol.

The CO-04-0307 PDX model was established for preclinical efficacy studies at WuXi AppTec. This PDX model was derived from an 82-year-old female Chinese CRC patient. A KRASG12C mutation in the PDX model CO-04-0307 was confirmed by whole exome sequencing and PCR sequencing. Mouse skin was cleaned with appropriate surgical scrub and alcohol over the right flank. Tumor fragments (15-30 mm³) harvested from the PDX model (FP5) were implanted subcutancously in the right flanks of mouse using an 18g trochar needle. 300 mice were implanted in this study. Animal health and tumor growth were monitored daily. Tumor volume was measured twice a week by caliper when tumors were palpable and measurable. When tumor volumes reached a mean of 155 mm³ (range of 96-259 mm³) at day 22 post subcutaneous implantation, tumor-bearing mice were randomized into different groups with 8 mice in each group. The randomization date was denoted as treatment day 0.

### Treatment

Treatment started on the day after randomization. The treatment start day was denoted as treatment day 1. Mice were dosed by oral administration of vehicle control solution or monotherapy treatments of Compound 1 at 30 mg/kg/dose BID or sotorasib at 100 mg/kg QD. An additional group received combination treatment of Compound 1 at 30 mg/kg/dose BID or sotorasib at 100 mg/kg QD. BID was dosed at an 8-hour interval. In addition to regular food and water supply. DictGel (Ready Jelly® 76A. Ready Biotechnology (ShenZhen) Co., Ltd.) was added in cages where at least two mice in a treatment group started showing >10% BWL at any measurement day. DietGel was supplied through the remaining study period after the addition of DietGel. Per this practice, mice in the Compound 1 at 30 mg/kg/dosc BID monotherapy treatment group and mice in the combination treatment group were supplied with DictGel food starting on treatment day 7. The study was terminated on treatment day 28 as defined in the study protocol.

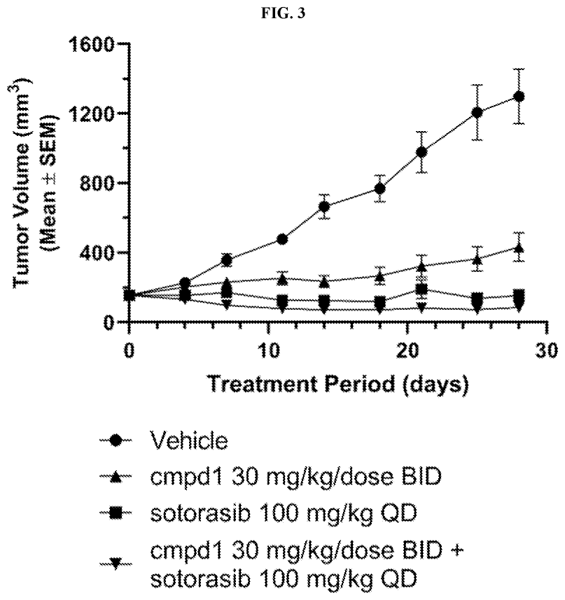

As illustrated by FIG. 3. Compound 1 and sotorasib demonstrate combination benefit in vivo in a KRAS^{G12C} Mutant CRC PDX model.

### Example 5: In Vivo KRAS^{G12C} Mutant CRC PDX model CO-04-0310 Assay

The vehicle/control article Propylene Glycol (PG) in deionized water, was prepared and stored under ambient conditions throughout the 28-day administration in mice.

The test article Compound 1 was prepared in vehicle of 0.5% Methyl Cellulose (MC) and 0.1% Tween 80 solution weekly and stored under ambient conditions. The combination agent sotorasib was prepared weekly in vehicle of 50% Polyethylene Glycol 400+50% Propylene Glycol w/w, and stored at 2-8° C.

Female Balb/c nude mice were between 6-8 weeks of age at the time of implantation. Mice were hosted in a special pathogen-free (SPF) environment of the vivarium facility and acclimated to their new environment for at least 3 days prior to initiation of any experiments according to IACUC protocol.

CO-04-0310 PDX model was established for preclinical efficacy study at WuXi AppTec. This PDX model was derived from an 82-year-old female Chinese CRC patient. A KRASG12C mutation in the PDX model CO-04-0310 was confirmed by whole exome sequencing and PCR sequencing. Mouse skin was cleaned with appropriate surgical scrub and alcohol over the right flank. Tumor fragments (15-30 mm³) harvested from the PDX model (FP5) were implanted subcutaneously in the right flanks of mouse using an 18g trochar needle. Totally 300 mice were implanted in this study. Animal health and tumor growth were monitored daily. Tumor volume was measured twice a week by caliper when tumors were palpable and measurable. When tumor volumes reached a mean of 157 mm³ (range of 95-240 mm³) at day 18 post subcutancous implantation, tumor-bearing mice were randomized into different groups with 8 mice in each group. The randomization date was denoted as treatment day 0.

### Treatment

Treatment started on the day after randomization. The treatment start day was denoted as treatment day 1. Mice were dosed by oral administration of vehicle control solution or monotherapy treatments of Compound 1 at 30 mg/kg/dose BID or sotorasib at 100 mg/kg QD. An additional group received combination treatment of Compound 1 at 30 mg/kg/dose BID or sotorasib at 100 mg/kg QD. BID was dosed at an 8-hour interval. In the combination group, sotorasib was dosed first and Compound 1 was dosed one hour later. In addition to regular food and water supply. DietGel (Ready Jelly R. 76A. Ready Biotechnology (ShenZhen) Co., Ltd.) was added in cages where at least two mice in a treatment group started showing >10% BWL at any measurement day. DietGel was supplied through the remaining study period after the addition of DietGel. Per this practice, mice in the Compound 1 at 30 mg/kg/dose BID monotherapy treatment group were supplied with DictGel food starting on treatment day 14. The study was terminated on treatment day 28 as defined in the study protocol.

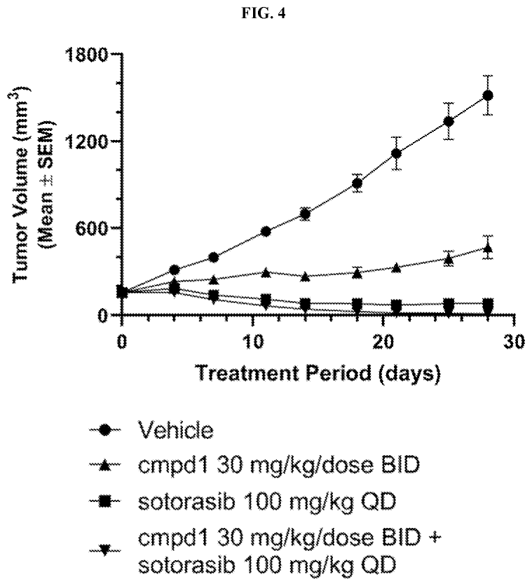

As illustrated by FIG. 4, Compound 1 and sotorasib demonstrate combination benefit in vivo in a KRAS^{G12C} Mutant CRC PDX model.

### Example 6: In vivo KRAS G12C mutant NSCLC PDX model LU-01-0046 assay

The vehicle/control article Propylene Glycol (PG) in deionized water, was prepared and stored under ambient conditions throughout the 28-day administration in mice.

The test article Compound 1 was prepared in vehicle of 0.5% Methyl Cellulose (MC) and 0.1% Tween 80 solution weekly and stored under ambient conditions. The combination agent sotorasib was prepared weekly in vehicle of 50% Polyethylene Glycol 400+50% Propylene Glycol w/w, and stored at 2-8° C.

Female Balb/c nude mice were between 6-8 weeks of age at the time of implantation. Mice were hosted in a special pathogen-free (SPF) environment of the vivarium facility and acclimated to their new environment for at least 3 days prior to initiation of any experiments according to IACUC protocol.

The LU-01-0046 PDX model was established for preclinical efficacy study at WuXi AppTec. This PDX model was derived from a 74-year-old male Chinese NSCLC patient. A KRAS^{G12C} mutation in the PDX model LU-01-0046 was confirmed by whole exome sequencing and PCR sequencing. Mouse skin was cleaned with appropriate surgical scrub and alcohol over the right flank. Tumor fragments (15-30 mm³) harvested from the PDX model (FP5) were implanted subcutaneously in the right flanks of mouse using an 18g trochar needle. 300 mice were implanted in this study. Animal health and tumor growth were monitored daily. Tumor volume was measured twice a week by caliper when tumors were palpable and measurable. When tumor volumes reached a mean of 191 mm³ (range of 84-321 mm³) at day 21 post subcutaneous implantation, tumor-bearing mice were randomized into different groups with 8 mice in each group. The randomization date was denoted as treatment day 0.

### Treatment

Treatment started on the day after randomization. The treatment start day was denoted as treatment day 1. Mice were dosed by oral administration of vehicle control solution or monotherapy treatments of Compound 1 at 30 mg/kg/dose BID or sotorasib at 100 mg/kg QD. An additional group received combination treatment of Compound 1 at 30 mg/kg/dose BID or sotorasib at 100 mg/kg QD. BID was dosed at an 8-hour interval. In the combination group, sotorasib was dosed first and Compound 1 was dosed one hour later.

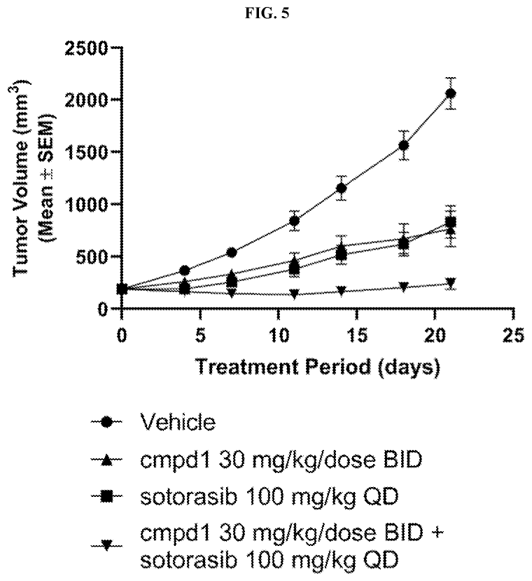

As illustrated by FIG. 5, Compound 1 and sotorasib demonstrate combination benefit in vivo in a KRAS^{G12C} mutant NSCLC PDX model.

1. A method of treating cancer in a subject in need thereof, the method comprising: administering to the subject in need thereof a therapeutically effective amount of
(i) compound 1: 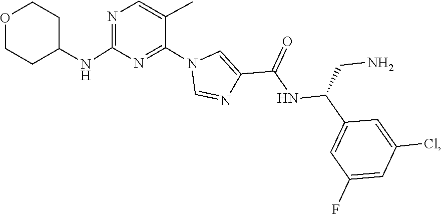 [SMILES: Cc1cnc(NC2CCOCC2)nc1-n1cnc(C(=O)N[C@H](CN)c2cc(F)cc(Cl)c2)c1]
or a pharmaceutically acceptable salt thereof; and
(ii) a KRAS G12C inhibitor.

2. The method of claim 1, wherein the KRAS G12C inhibitor is adagrasib, ARS-3248, BBP-454, BI 1701963, GDC-6036, sotorasib, or tipifarnib.

3. The method of claim 1 or 2, wherein the KRAS G12C inhibitor is sotorasib.

4. The method of claim 2 or 3, wherein sotorasib is administered in an amount that is about 960 mg/day.

5. The method of claim 1 or 2, wherein the KRAS G12C inhibitor is adagrasib.

6. The method of claim 2 or 5, wherein adagrasib is administered in an amount that is about 1200 mg/day.

7. A method of treating cancer in a subject in need thereof, the method comprising: administering to the subject in need thereof a therapeutically effective amount of
(i) compound 1: 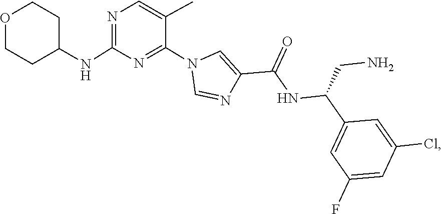 [SMILES: Cc1cnc(NC2CCOCC2)nc1-n1cnc(C(=O)N[C@H](CN)c2cc(F)cc(Cl)c2)c1]
or a pharmaceutically acceptable salt thereof; and
(ii) sotorasib.

8. A method of treating cancer in a subject in need thereof, the method comprising: administering to the subject in need thereof a therapeutically effective amount of
(i) compound 1: 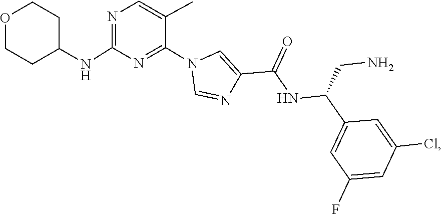 [SMILES: Cc1cnc(NC2CCOCC2)nc1-n1cnc(C(=O)N[C@H](CN)c2cc(F)cc(Cl)c2)c1]
or a pharmaceutically acceptable salt thereof; and
(ii) adagrasib.

9. The method of any one of claims 1-8, wherein the pharmaceutically acceptable salt of compound 1 is the mandelic acid salt.

10. The method of any one of claims 1-8, wherein the cancer is a mitogen-activated protein kinase (MAPK) pathway driven cancer.

11. The method of any one of claims 1-8, wherein the cancer is a BRAF-driven cancer, HRAS-driven cancer, or a NRAS-driven cancer.

12. The method of any one of claims 1-8, wherein the cancer comprises at least one cancer cell driven by deregulated ERK.

13. The method of any one of claims 1-8, wherein the cancer has at least one mutation in RAS.

14. The method of any one of claims 1-8, wherein the cancer has at least one mutation in RAF.

15. The method of any one of claims 1-8, wherein the cancer has at least one mutation in MEK.

16. The method of any one of claims 1-8, wherein the cancer has a G12C KRAS mutation.

17. The method of any one of claims 1-8, wherein the cancer has a G12D KRAS mutation.

18. The method of any one of claims 1-8, wherein the cancer has a G12S KRAS mutation.

19. The method of any one of claims 1-8, wherein the cancer has a G12V KRAS mutation.

20. The method of any one of claims 1-8, wherein the cancer has a G13D KRAS mutation.

21. The method of any one of claims 1-8, wherein the cancer has a Q16H KRAS mutation.

22. The method of any one of claims 1-8, wherein the cancer has a Q16K KRAS mutation.

23. The method of any one of claims 1-8, wherein the cancer has a Q61R NRAS mutation.

24. The method of any one of claims 1-8, wherein the cancer is a BRAF V600E or V600K mutant tumor.

25. The method of any one of claims 1-8, wherein the cancer is a MAPKm/MAPKi-naïve pan cancer.

26. The method of any one of claims 1-8, wherein the cancer comprises one or more EGFR mutation selected from the group consisting of EGFR gene copy gain, EGFR gene amplification, chromosome 7 polysomy, L858R, exon 19 deletions/insertions, L861Q, G719C, G719S, G719A, V765A, T783A, exon 20 insertions, EGFR splice variants (Viii, Vvi, and Vii), A289D, A289T, A289V, G598A, G598V, T790M, and C797S.

27. The method of any one of claims 1-8, wherein the cancer comprises one or more EGFR mutation selected from the group consisting of L858R, exon 19 deletion, and T790M.

28. The method of any one of claims 1-27, wherein the cancer is a solid tumor.

29. The method of any one of claims 1-28, wherein the cancer is non-small cell lung cancer (NSCLC), melanoma, pancreatic cancer, salivary gland tumor, thyroid cancer, colorectal cancer (CRC), or esophageal cancer.

30. The method of any one of claims 1-28, wherein the cancer is non-small cell lung cancer (NSCLC).

31. The method of claim 30, wherein the NSCLC is an EGFR mutant NSCLC.

32. The method of claim 30, wherein the NSCLC is a KRAS G12C mutant NSCLC.

33. The method of claim 30, wherein the NSCLC is a KRAS G12D mutant NSCLC.

34. The method of claim 30, wherein the NSCLC is a KRAS G12S mutant NSCLC.

35. The method of claim 30, wherein the NSCLC is a KRAS G12V mutant NSCLC.

36. The method of claim 30, wherein the NSCLC is a KRAS G13D mutant NSCLC.

37. The method of claim 30, wherein the NSCLC is a KRAS Q61H mutant NSCLC.

38. The method of claim 30, wherein the NSCLC is a KRAS Q61K mutant NSCLC.

39. The method of claim 30, wherein the NSCLC is a NRAS Q61R mutant NSCLC.

40. The method of claim 30, wherein the cancer is a MAPKm/MAPKi-naïve NSCLC.

41. The method of claim 30, wherein the cancer is a BRAFi-treated V600 NSCLC.

42. The method of claim 30, wherein the cancer is a KRAS-treated G12C NSCLC.

43. The method of claim 30, wherein the cancer is a KRAS-treated G12D NSCLC.

44. The method of claim 30, wherein the cancer is a KRAS-treated G12S NSCLC.

45. The method of claim 30, wherein the cancer is a KRAS-treated G12V NSCLC.

46. The method of claim 30, wherein the cancer is a KRAS-treated G13D NSCLC.

47. The method of claim 30, wherein the cancer is a KRAS-treated Q61H NSCLC.

48. The method of claim 30, wherein the cancer is a KRAS-treated Q61K NSCLC.

49. The method of claim 30, wherein the cancer is a NRAS-treated Q61R NSCLC.

50. The method of any one of claims 1-28, wherein the cancer is pancreatic cancer.

51. The method of claim 50, wherein the cancer is a MAPKm/MAPKi-naïve pancreatic cancer.

52. The method of any one of claims 1-28, wherein the cancer is melanoma.

53. The method of claim 52, wherein the melanoma is a BRAF V600E or V600K mutant tumor.

54. The method of claim 52, wherein the cancer is a BRAFi-treated V600 melanoma.

55. The method of any one of claims 1-28, wherein the cancer is salivary gland tumor.

56. The method of any one of claims 1-28, wherein the cancer is thyroid cancer.

57. The method of any one of claims 1-28, wherein the cancer is colorectal cancer (CRC).

58. The method of claim 57, wherein the CRC is a BRAF V600E CRC.

59. The method of claim 57, wherein the CRC is a KRAS mutant CRC.

60. The method of claim 59, wherein the CRC is a KRAS G12C mutant CRC.

61. The method of claim 59, wherein the CRC is a KRAS G12D mutant CRC.

62. The method of claim 59, wherein the CRC is a KRAS G12S mutant CRC.

63. The method of claim 59, wherein the CRC is a KRAS G12V mutant CRC.

64. The method of claim 59, wherein the CRC is a KRAS G13D mutant CRC.

65. The method of claim 59, wherein the CRC is a KRAS Q61H mutant CRC.

66. The method of claim 59, wherein the CRC is a KRAS Q61K mutant CRC.

67. The method of claim 57, wherein the CRC is a NRAS mutant CRC.

68. The method of claim 67, wherein the CRC is a NRAS Q61R mutant CRC.

69. The method of any one of claims 1-28, wherein the cancer is esophageal cancer.

70. The method of any one of claims 1-69, wherein compound 1, or a pharmaceutically acceptable salt thereof, is administered in an amount that is between about 25 mg/day and about 300 mg/day.

71. The method of any one of claims 1-70, wherein compound 1, or a pharmaceutically acceptable salt thereof, is administered in an amount that is between 25 mg/day and 150 mg/day.

72. The method of any one of claims 1-71, wherein compound 1, or a pharmaceutically acceptable salt thereof, is administered in an amount that is about 25 mg/day, about 50 mg/day, about 75 mg/day, about 100 mg/day, about 125 mg/day about 150 mg/day, about 175 mg/day, about 200 mg/day, about 225 mg/day, or about 250 mg/day.

73. The method of any one of claims 1-72, wherein compound 1, or a pharmaceutically acceptable salt thereof, is administered in an amount that is about 25 mg/day, about 50 mg/day, about 100 mg/day, or about 150 mg/day.

74. The method of any one of claims 1-71, wherein compound 1, or a pharmaceutically acceptable salt thereof, is administered in an amount that is about 250 mg/day.

75. The method of any one of claims 1-74, wherein compound 1, or a pharmaceutically acceptable salt thereof, is administered once a day (QD).

76. The method of any one of claims 1-74, wherein compound 1, or a pharmaceutically acceptable salt thereof, is administered twice a day (BID).

77. The method of any one of claims 1-74, wherein compound 1, or a pharmaceutically acceptable salt thereof, is administered three times a day (TID).

78. The method of any one of claims 1-77, wherein compound 1, or a pharmaceutically acceptable salt thereof, is administered once a week.

79. The method of any one of claims 1-77, wherein compound 1, or a pharmaceutically acceptable salt thereof, is administered twice a week.

80. The method of any one of claims 1-69, wherein compound 1, or a pharmaceutically acceptable salt thereof, is administered in an amount that is between about 25 mg and about 300 mg twice a day, once a week (BID-QW).

81. The method of any one of claims 1-69, wherein compound 1, or a pharmaceutically acceptable salt thereof, is administered in an amount that is between about 25 mg and about 250 mg twice a day, once a week (BID-QW).

82. The method of any one of claims 1-69, wherein compound 1, or a pharmaceutically acceptable salt thereof, is administered in an amount that is between about 25 mg and about 150 mg twice a day, once a week (BID-QW).

83. The method of any one of claims 1-69, wherein compound 1, or a pharmaceutically acceptable salt thereof, is administered in an amount that is about 25 mg, 50 mg, about 75 mg, about 100 mg, about 125 mg, about 150 mg, about 175 mg, about 200 mg, about 225 mg, or about 250 mg twice a day, once a week (BID-QW).

84. The method of any one of claims 1-69, wherein compound 1, or a pharmaceutically acceptable salt thereof, is administered in an amount that is about 25 mg, 50 mg, about 100 mg, about 125 mg, or about 150 mg twice a day, once a week (BID-QW).

85. The method of any one of claims 1-69, wherein compound 1, or a pharmaceutically acceptable salt thereof, is administered in an amount that is about 125 mg twice a day, once a week (BID-QW).

86. The method of any one of claims 1-85, wherein compound 1, or a pharmaceutically acceptable salt thereof, is administered for at least one 28-day cycle.

87. The method of any one of claims 1-86, wherein compound 1, or a pharmaceutically acceptable salt thereof, is administered on day 1, day 8, day 15, and day 22 of a 28-day cycle.

88. The method of any one of claims 1-86, wherein compound 1, or a pharmaceutically acceptable salt thereof, is administered on day 1, day 8, day 15 of a 28-day cycle.

89. The method of any one of claims 1-88, wherein compound 1, or a pharmaceutically acceptable salt thereof, is administered orally.

90. The method of any one of claims 1-89, wherein the method further comprises administering an additional MAPK pathway inhibitor.

91. The method of claim 90, wherein the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFRIR inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor.

92. The method of claim 90, wherein the additional MAPK pathway inhibitor is a KRAS inhibitor.

93. The method of claim 90, wherein the additional MAPK pathway inhibitor is a BRAF inhibitor.

94. The method of claim 90, wherein the additional MAPK pathway inhibitor is a EGFR inhibitor.

95. The method of claim 90, wherein the additional MAPK pathway inhibitor is a CDK4/6.

96. The method of claim 90, wherein the additional MAPK pathway inhibitor is a FLT3 inhibitor.

97. The method of claim 90, wherein the additional MAPK pathway inhibitor is adagrasib, afatinib, ASTX029, binimetinib, cetuximab, cobimetinib, dabrafenib, dacomitinib, encorafenib, erlotinib, gefitinib, gilteritinib, lapatinib, LTT462, LY3214996, necitumumab, neratinib, nimotuzumab, osimertinib, palbociclib, panitumumab, selumetinib, sotorasib, tramctinib, ulixertinib, and vandetanib.

98. The method of claim 90, wherein the additional MAPK pathway inhibitor is cetuximab.

99. The method of claim 90, wherein the additional MAPK pathway inhibitor is dabrafenib.

100. The method of claim 90, wherein the additional MAPK pathway inhibitor is encorafenib.

101. The method of claim 90, wherein the additional MAPK pathway inhibitor is gilteritinib.

102. The method of claim 90, wherein the additional MAPK pathway inhibitor is palbociclib.

103. The method of claim 90, wherein the additional MAPK pathway inhibitor is panitumumab.
